(19) United States
(12) Patent Application Publication
(10) Pub. No.: US 2023/0165509 A1
(43) Pub. Date: Jun. 1, 2023

(54) METHODS AND DEVICES FOR VIRTUALLY RECONSTRUCTING BRAIN-WIDE NEURAL ACTIVITY FROM LOCAL ELECTROPHYSIOLOGICAL RECORDINGS

(71) Applicant: The Regents of the University of California, Oakland, CA (US)

(72) Inventors: Duygu Kuzum, La Jolla, CA (US); Xin Liu, La Jolla, CA (US); Takaki Komiyama, La Jolla, CA (US); Chi Ren, La Jolla, CA (US)

(21) Appl. No.: 18/060,439

(22) Filed: Nov. 30, 2022

Related U.S. Application Data
(60) Provisional application No. 63/264,660, filed on Nov. 30, 2021.

(51) Int. Cl. A61B 5/37; A61B 5/384; G16H 50/50

(52) U.S. Cl. CPC A61B 5/37; A61B 5/384; G16H 50/50

(57) ABSTRACT

Methods and devices for computationally constructing brain potentials across whole brain using electrocorticography signals recorded from a small region on the brain surface are disclosed. In some embodiments of the disclosed technology, a method includes obtaining a plurality of locally recorded surface potentials from a plurality of first cortical areas of a brain surface; and performing a virtual reconstruction of an average brain activity for individual cortical areas and a pixel-level cortex-wide brain activity for a plurality of cortical areas of the brain surface including the plurality of first cortical areas based on the plurality of locally recorded surface potentials.

### PRIORITY CLAIM AND CROSS-REFERENCE TO RELATED APPLICATIONS

This patent document claims the priority and benefits of U.S. Provisional Application No. 63/264,660, titled “METHODS AND DEVICES FOR VIRTUALLY RECONSTRUCTING BRAIN-WIDE NEURAL ACTIVITY FROM LOCAL ELECTROPHYSIOLOGICAL RECORDINGS” filed on Nov. 30, 2021. The entire content of the aforementioned patent application is incorporated by reference as part of the disclosure of this patent document.

### STATEMENT REGARDING FEDERALLY SPONSORED RESEARCH OR DEVELOPMENT

This invention is made with government support under NIH R21 EB026180 awarded by the National Institute of Health (NIH) and NSF ECCS-1752241 awarded by the National Science Foundation (NSF). The government has certain rights in the invention.

### TECHNICAL FIELD

The technology and implementations disclosed in this patent document generally relate to neural activity imaging.

### BACKGROUND

As an important tool for electrophysiological recordings, neural electrodes implanted on the brain surface have been instrumental in basic neuroscience research to study large-scale neural dynamics in various cognitive processes, such as sensorimotor processing as well as learning and memory. In clinical settings, neural recordings have been adopted as a standard tool to monitor the brain activity in epilepsy patients before surgery for detection and localization of epileptogenic zones initiating seizures and functional cortical mapping. Neural activity recorded from the brain surface exhibits rich information content about the collective neural activities reflecting the cognitive states and brain functions. For the interpretation of surface potentials in terms of their neural correlates, most research has focused on local neural activities.

### SUMMARY

The disclosed technology can be implemented in some embodiments to provide methods and devices for virtually reconstructing brain-wide neural activity from local electrophysiological recordings using a recurrent neural network.

In some implementations of the disclosed technology, a method includes obtaining a plurality of electrical signals from an array of electrodes implanted on a plurality of first cortical local regions of a brain at a plurality of frequency bands during a first time interval; determining, based on the plurality of electrical signals, an average brain activity for individual cortical local regions corresponding to the plurality of first cortical local regions and a plurality of second cortical local regions different from the plurality of first cortical local regions; and reconstructing a cortex-wide brain activity with pixel-level spatial resolution including a brain activity for the first and second cortical local regions at a first point in time during the first time interval using weighting scores of a plurality of independent components that are obtained based on the plurality of electrical signals.

In some implementations of the disclosed technology, a method includes obtaining a plurality of locally recorded surface potentials from a plurality of first cortical areas of a brain surface; and performing a virtual reconstruction of an average brain activity for individual cortical areas and a pixel-level cortex-wide brain activity for a plurality of cortical areas of the brain surface including the plurality of first cortical areas based on the plurality of locally recorded surface potentials.

In some implementations of the disclosed technology, a device includes an array of electrodes configured to be implanted on a plurality of first cortical local regions of a brain; a memory to store instructions for performing a virtual reconstruction of an activity of the brain; and a processor in communication with the memory, wherein the instructions upon execution by the process cause the processor to: obtain a plurality of electrical signals from the array of electrodes implanted on a plurality of first cortical local regions of the brain at a plurality of frequency bands during a first time interval; determine, based on the plurality of electrical signals, an average brain activity for individual cortical local regions corresponding to the plurality of first cortical local regions and a plurality of second cortical local regions different from the plurality of first cortical local regions; and reconstruct a cortex-wide brain activity with pixel-level spatial resolution including a brain activity for the first and second cortical local regions at a first point in time during the first time interval using weighting scores of a plurality of independent components that are obtained based on the plurality of electrical signals using a spatial independent component analysis.

In some implementations of the disclosed technology, a method includes obtaining a plurality of locally recorded surface potentials from an array of electrodes implanted on a plurality of cortical areas of a brain surface, and performing a virtual reconstruction of a cortex-wide brain activity using the plurality of locally recorded surface potentials.

The above and other aspects and implementations of the disclosed technology are described in more detail in the drawings, the description and the claims.

### BRIEF DESCRIPTION OF THE DRAWINGS

FIG. 1A shows schematic of the multimodal experimental setup combining neural recordings using transparent graphene electrodes and wide-field calcium imaging. FIG. 1B shows an example field of view of wide-field calcium imaging during experiment. FIG. 1C shows imaged cortical regions based on Allen Brain Atlas. FIG. 1D shows wide-field fluorescence activity during 10-s long recordings. FIG. 1E shows fluorescence activity for different cortical regions, the simultaneously recorded neural signals for a 3-s time interval, and their power at three frequency bands. FIG. 1F shows the average power increase at different frequency bands for all the ECoG channels during activations of cortical areas right beneath the array.

FIG. 2 shows an example of a decoding model implemented based on some embodiments of the disclosed technology.

FIG. 3A shows decoded and ground truth ΔF/F activity of different cortical regions in the contralateral and ipsilateral hemispheres for one mouse. FIG. 3B shows decoding performance evaluated for different cortical regions in the contralateral and ipsilateral hemispheres using different frequency bands. FIG. 3C shows decoding performance for different cortical regions in the contralateral and ipsilateral hemispheres evaluated as a function of distance. FIG. 3D shows decoding performance for different cortical regions in the contralateral and ipsilateral hemispheres using all the frequency bands. FIG. 3E shows decoding performance evaluated for different cortical regions using different number of channels.

FIG. 4A shows a profile plot of pixel intensity for enriched and non-enriched samples of dye deposited on paper. FIG. 4B shows an image sequence of dye localization in paper with hydrophobically patterned barriers indicated by black outlines. FIG. 4C shows reconstructed and ground truth cortex-wide ΔF/F activity for 4 different time intervals. FIG. 4D shows decoding performance evaluated for different independent components (ICs) for one recording session.

FIG. 4E shows decoding performance evaluated at pixel-level for all the cortical regions in the ipsilateral and contralateral hemispheres. FIG. 4F shows pixel-wise decoding performance evaluated at individual cortical regions and displayed as a function of distance to the array.

FIG. 5A shows identified principal components for individual recording sessions, showing different cortical co-activation patterns. FIG. 5B shows the proportion of variance explained by each principal component for individual recording sessions.

FIG. 6 shows independent components (ICs) for cortical activity for all the animals. Similar cortical functional modules and blood vessel activities are identified across different animals.

FIG. 7A shows the ground truth activity and the decoded activity. FIG. 7B shows Decoding performance evaluated for different cortical regions in the contralateral and ipsilateral hemispheres using shuffled data from different frequency bands. FIG. 7C shows Decoding performance for different cortical regions in the contralateral and ipsilateral hemispheres using shuffled data from all the frequency bands, but different numbers of recording channels. FIG. 7D shows decoded and ground truth weighting scores of the observed cortex-wide activity onto the 10 ICs shown in FIG. 4A using shuffled data. FIG. 7E shows decoding performance evaluated at pixel-level for all the cortical regions in the ipsilateral and contralateral hemispheres using shuffled data.

FIG. 8A shows the decoding performance evaluated at 30 s sliding window for the data shown in FIG. 3A. FIG. 8B shows the decoding performance of individual cortical regions between movement and rest phases.

FIG. 9 shows comparison of the decoding performance using High-gamma power band vs. other frequency bands for ipsilateral cortical regions.

FIG. 10 shows comparison of the decoding performance using High-gamma power band vs. other frequency bands for contralateral cortical regions.

FIG. 11A shows the decoding performance of ipsilateral cortices plotted against distance rank to the array for different frequency bands. FIG. 11B shows the slope of the decoding performance vs. distance for different frequency bands.

FIG. 12 shows the correlation between the activity of each ICs and the PC1.

FIG. 13 shows decoding performance of the IC scores for all the sessions recorded for all the animals. Each dot marks one cross-validated correlation for one fold data.

FIG. 14 shows correlation of ΔF/F activity from different cortical regions in all the mice.

FIG. 15 shows an example method for reconstructing a cortex-wide brain activity based on some embodiments of the disclosed technology.

FIG. 16 shows an example of a virtual reconstruction method of a cortex-wide brain activity based on some embodiments of the disclosed technology.

### DETAILED DESCRIPTION

Disclosed are methods, materials, articles of manufacture and devices that pertain to computationally constructing brain potentials across whole brain using electrocorticography signals recorded from a small region on the brain surface.

From basic neuroscience research to clinical treatments and neural engineering, electrocorticography (ECoG) has been widely used to record surface potentials to evaluate brain function and develop neuroprosthetic devices. However, the requirement of invasive surgeries for implanting ECoG arrays significantly limits the coverage of different cortical regions, preventing simultaneous recordings from spatially distributed cortical networks. This rich information content of surface potentials encoded for the large-scale cortical activity remains unexploited and little is known on how local surface potentials are correlated with the spontaneous neural activities of distributed large-scale cortical networks.

The disclosed technology can be implemented in some embodiments to decode cortex-wide brain activity from local recordings of neural potentials.

In some embodiments of the disclosed technology, a cortex-wide activity can be inferred from locally recorded ECoG signals. In some implementations, graphene electrodes can be used to collect training data for the neural network algorithm. For example, transparent graphene microelectrode arrays are implanted over the mouse somatosensory cortex and ECoG recordings and wide-field calcium imaging of the dorsal cortex are performed simultaneously in awake, head-fixed mice. By developing a recurrent neural network model using locally recorded ECoG signals as inputs, virtual imaging of the averaged spontaneous activity from multiple cortical areas and the cortex-wide activity with pixel-level spatial resolution can be demonstrated.

The disclosed technology can be implemented in some embodiments to infer the cortex-wide brain activity based on the information content of the local neural potentials recorded from brain surface. In some implementations, brain potentials across whole brain can be computationally constructed by using electrocorticography signals recorded from a small region on the brain surface.

The disclosed technology can be implemented in some embodiments to allow large area mapping of neural dysfunctions and neurological disorders without requiring extremely invasive neural surgeries. Virtual implants will use data from clinical ECoG recordings on brain surface that are routinely performed in patients before brain surgeries and compute the brain potentials across the whole cortex. Neural circuit dysfunctions are the cause of most neurological diseases, including epilepsy, Parkinson's disease, dystonia, depression and schizophrenia. For example, when applied to epilepsy, virtual implants will precisely determine the exact coordinates of the neuronal population generating seizures, unlike conventional local recordings resulting in low success rate (50%) for epilepsy surgeries. That will greatly impact the outcome and success rate of brain surgeries. Furthermore, brain-computer interfaces (BCI) have shown great promise for tetraplegia (paralysis), but penetrating microelectrodes cause extensive tissue damage limiting decoding ability and the lifetime of prosthetics less than a year. Virtual implants will enable less invasive long-term BCI by enabling decoding of spike activity from surface ECoG arrays. Virtual Implants will lead us to new findings on neural dynamics that is unattainable otherwise and facilitate development of targeted treatments for neurological disorders affecting one billion people worldwide.

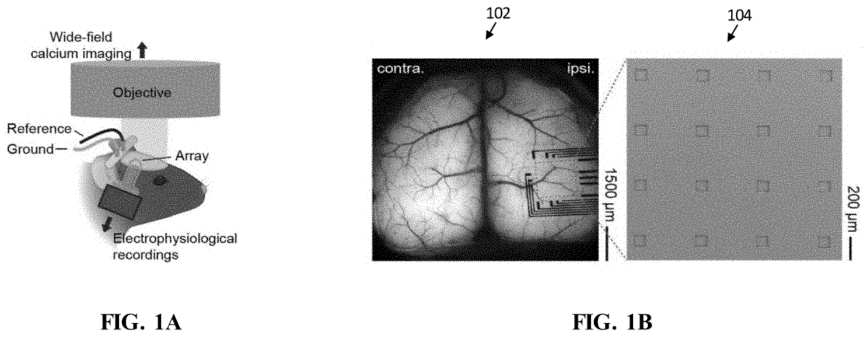

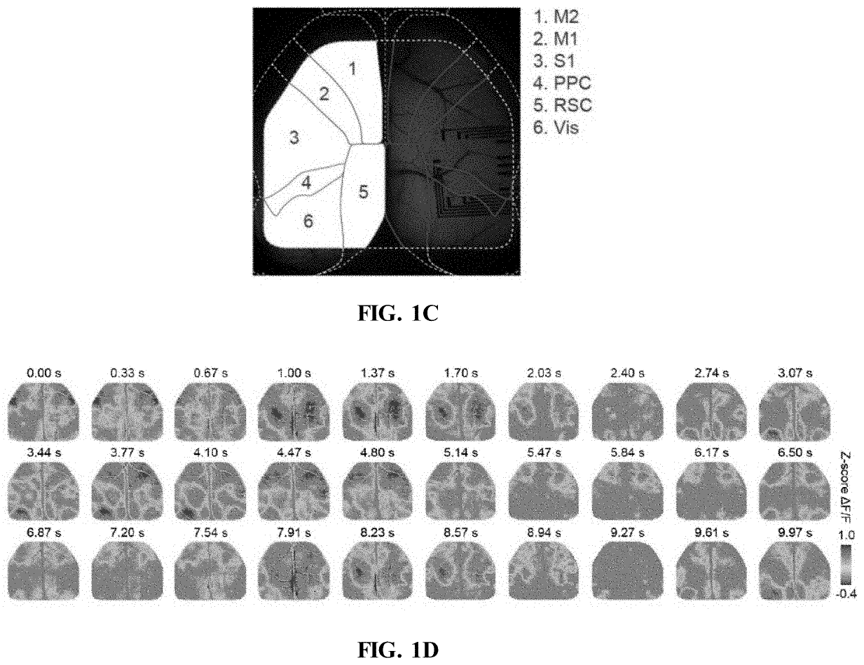

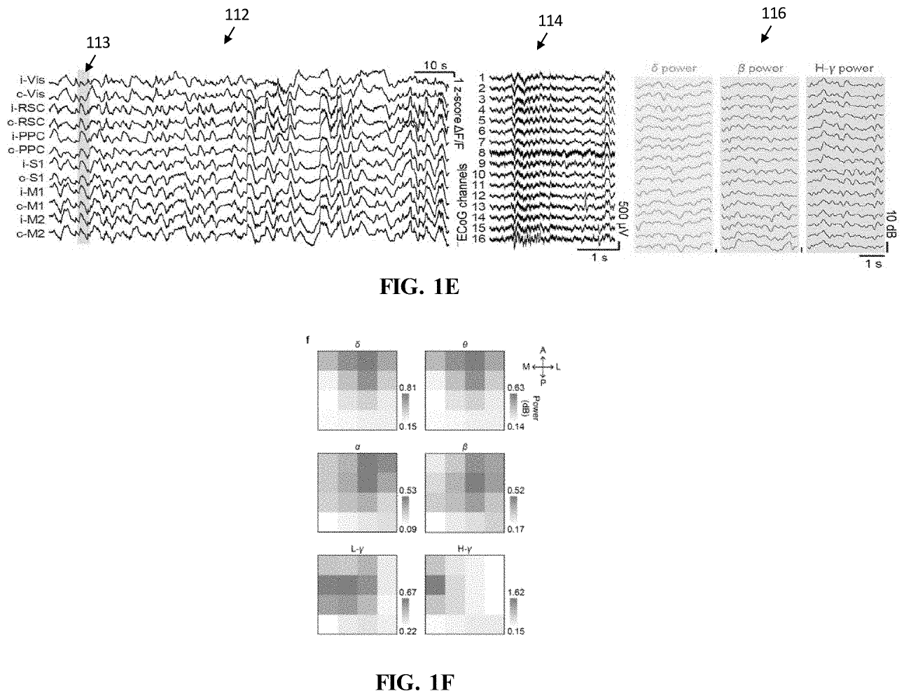

FIGS. 1A-1E show simultaneous multimodal wide-field calcium imaging and surface potential recordings (e.g., ECoG recordings). FIG. 1A shows schematic of the multimodal experimental setup combining neural recordings using transparent graphene electrodes and wide-field calcium imaging. FIG. 1B shows an example field of view of wide-field calcium imaging during experiment (102). Clear area at the center of the transparent array includes 16 graphene electrodes, whose scanning electron microscope image is shown on the right (104). FIG. 1C shows imaged cortical regions based on Allen Brain Atlas. M2 indicates secondary motor cortex, M1 indicates primary motor cortex, S1 indicates primary somatosensory cortex, PPC indicates posterior parietal cortex, RSC indicates retrosplenial cortex, and Vis indicates visual cortex. FIG. 1D shows wide-field fluorescence activity during 10 s long recordings, showing the diverse spontaneous activity across the mouse cortex. FIG. 1E shows fluorescence activity for different cortical regions (112), the simultaneously recorded neural signals (114) for a 3 s time interval (113), and their power at three frequency bands (δ: 1-4 Hz, β: 15-30 Hz, H_{γ}: 61-200 Hz, right three columns, 116). FIG. 1F shows the average power increase at different frequency bands for all the ECoG channels during activations of cortical areas right beneath the array (S1). The power increase exhibits a diverse spatial distribution for different frequency bands.

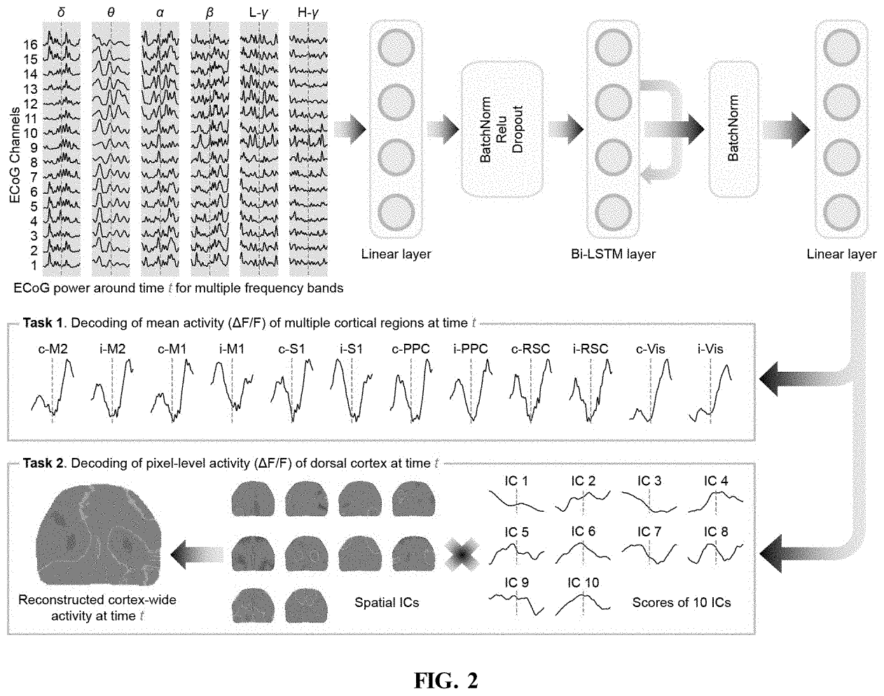

FIG. 2 shows schematic of the decoding model. Signal power (e.g., ECoG power) from different channels during time interval [t−1.5 s, t+1.5 s] (90 time steps) is used to decode the cortical activity at time point t. The decoding neural network model consists of a sequential stacking of a linear hidden layer, one bidirectional long short-term memory (Bi-LSTM) layer and a linear readout layer. For the task of decoding the mean ΔF/F activity from multiple cortical regions, the final linear readout layer directly outputs the activities of 12 cortical regions at time t. For the task of decoding the pixel-level cortex-wide brain activity, the final linear readout layer outputs the weighting scores for all the ICs at time t, from which the cortex-wide brain activity at time t is reconstructed.

In some implementations, the network takes the ECoG power from different ECoG channels during time interval [t−Δt, t+Δt] as the input. The output of multilayer perceptron (MLP) layer is fed into a single layer LSTM network with hidden units. Then the output of the LSTM network is flattened and fed into another MLP layer. For the task of decoding the ΔF/F activity from multiple cortical regions, the final MLP layer simultaneously outputs the ΔF/F activity for 12 cortical areas. For the task of decoding the whole brain ΔF/F activity, the final MLP layer outputs the scores on the chosen principal components, from which the whole brain image is reconstructed.

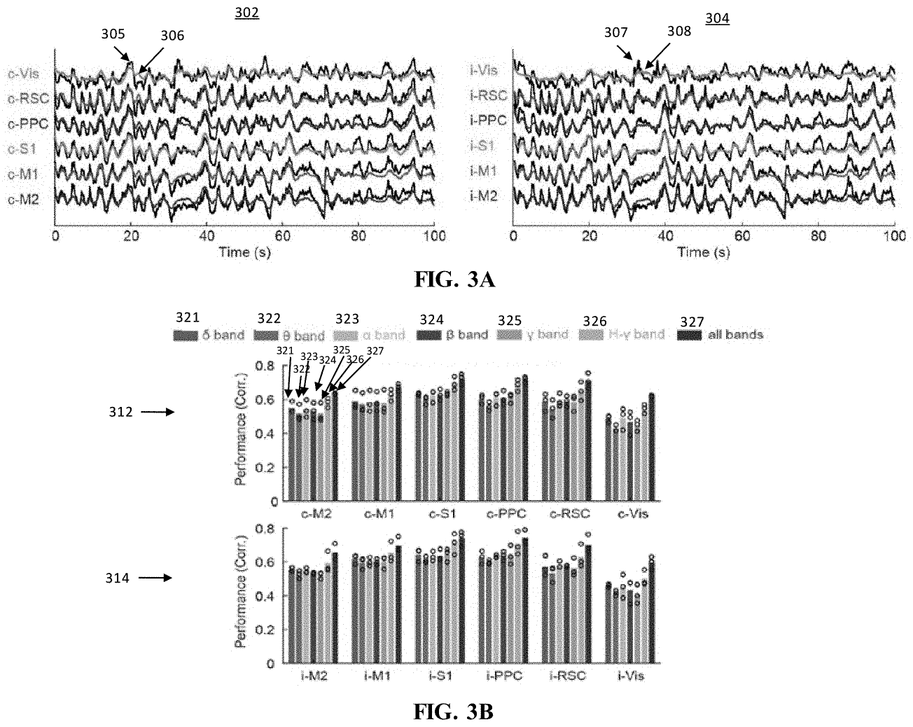

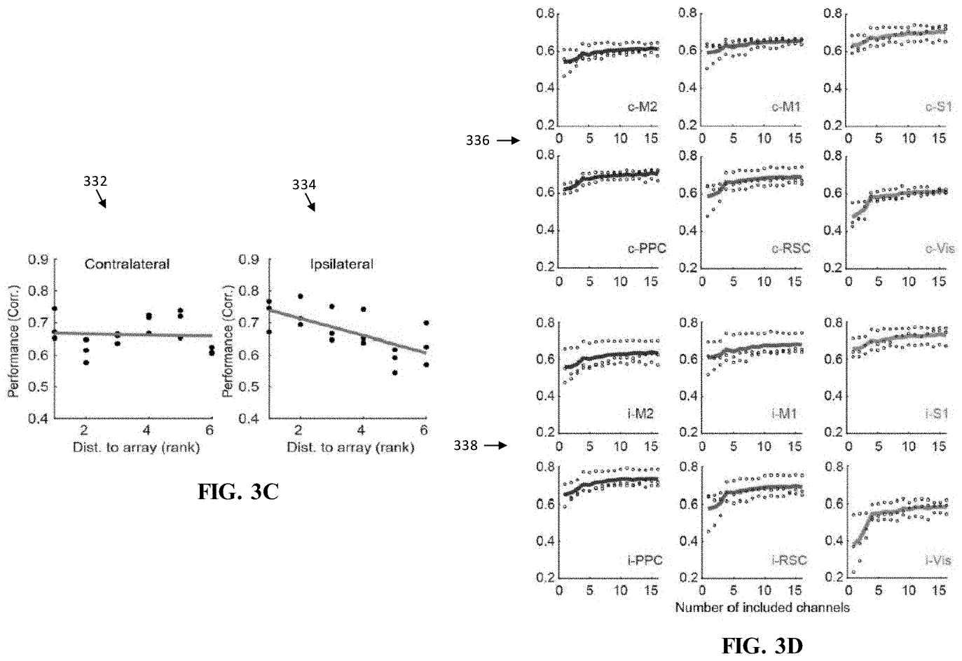

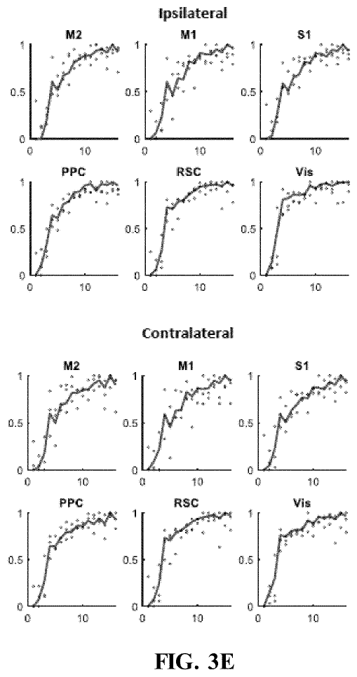

FIGS. 3A-3D show decoding of the activities of multiple cortical regions. FIG. 3A shows decoded (306, 308) vs. ground truth (305, 307) ΔF/F activity of different cortical regions in the contralateral (302) and ipsilateral (304) hemispheres for one mouse. FIG. 3B shows decoding performance evaluated for different cortical regions in the contralateral (312) and ipsilateral (314) hemispheres using different frequency bands (δ: 1-4 Hz (321), θ: 4-7 Hz (322), α: 8-15 Hz (323), β: 15-30 Hz (324), γ: 31-59 Hz (325), H_{γ}: 61-200 Hz (326), and all six frequency bands (327)). Each dot marks the mean correlation evaluated by ten-fold cross-validation using the data recorded from one mouse. FIG. 3C shows decoding performance for different cortical regions in the contralateral (332) and ipsilateral (334) hemispheres evaluated as a function of distance (rank orders). Each dot is the mean correlation for one mouse given by ten-fold cross-validation. For ipsilateral hemisphere, the decoding performance decreases as the distance rank to the electrode array increases (ρ=−0.676, P=0.002, n=18). For contralateral hemisphere, no such correlation is observed (ρ=−0.163, P=0.519, n=18). Distances from the center of the array to the center of each cortical region: i-M2 3.63 mm, i-M1 2.65 mm, i-S1 0.98 mm, i-PPC 0.7 mm, i-RSC 2.36 mm, i-Vis 2.49 mm, c-M2 5.01 mm, c-M1 5.53 mm, c-S1 5.96 mm, c-PPC 5.37 mm, c-RSC 3.83 mm, c-Vis 6.32 mm. FIG. 3D shows decoding performance for different cortical regions in the contralateral (336) and ipsilateral (338) hemispheres using all the frequency bands, but different numbers of recording channels. Each dot marks the mean ten-fold cross-validated correlation over all the recording sessions for one mouse. Each line is the mean correlation averaged across three mice. For all the cortical regions, the decoding performance increases as more recording channels are included (P<0.05, n=48, FDR correction). FIG. 3E shows decoding performance evaluated for different cortical regions using different number of channels. Each line is the correlation for one cortical region averaged across 3 mice. The error bar marks the SEM across 3 mice for a specific number of channels. For all the cortical regions, the correlation between the performance and the number of channels is significant (P<0.05, FDR correction). This is the normalized version. For each mouse, the correlation is normalized to [0,1] separately.

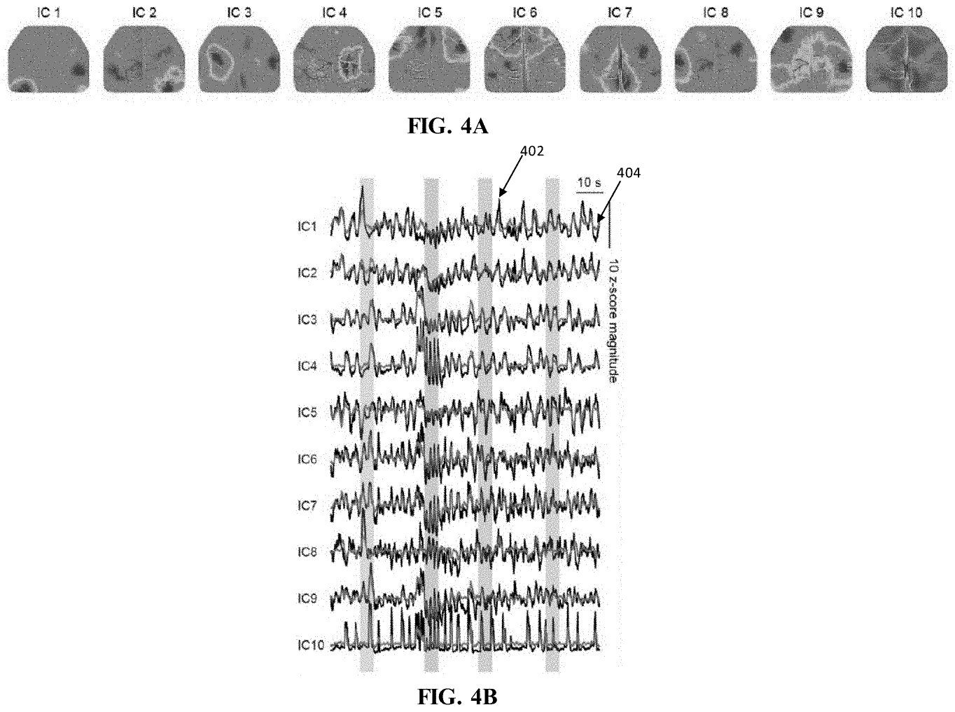

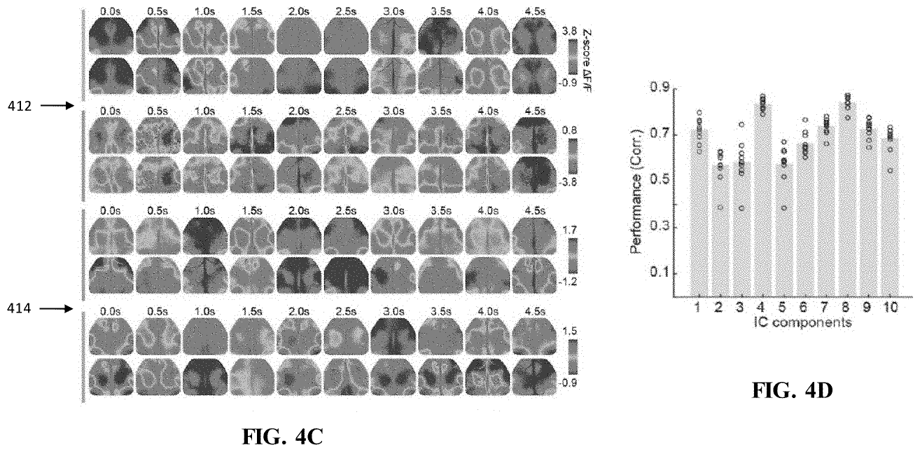

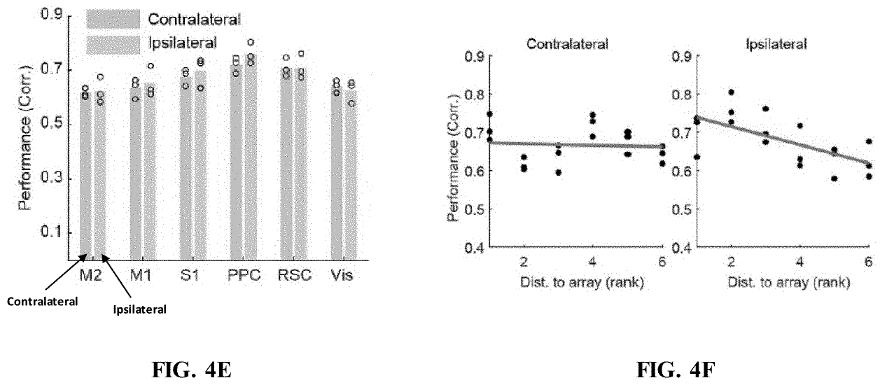

FIGS. 4A-4F show decoding of the pixel-level cortex-wide brain activity. FIG. 4A shows identified ICs (e.g., 10 ICs) for the cortical activities recorded in one mouse, showing different functional modules of the cortical activity (IC 1-9) and the blood vessel activity (IC 10). FIG. 4B shows decoded (404) and ground truth (402) weighting scores of the observed cortex-wide activity onto the ten ICs shown in FIG. 4A. FIG. 4C shows reconstructed (top rows, 412) and ground truth (bottom rows, 414) cortex-wide ΔF/F activity for four different time intervals, each lasting for 5 s, as indicated with different colors in FIG. 4B. For visualization, the reconstructed and true cortex-wide brain activity are shown for every 0.5 s. FIG. 4D shows decoding performance evaluated for different ICs for one recording session. Each dot marks the decoding performance evaluated on one fold during the ten-fold cross-validation. The weighting scores for all the ten ICs may be successfully decoded. FIG. 4E shows decoding performance evaluated at pixel-level for all the cortical regions in the ipsilateral and contralateral hemispheres. Each dot marks the mean ten-fold cross-validated correlation for individual pixels of one specific cortical region from one mouse. FIG. 4F shows pixel-wise decoding performance evaluated at individual cortical regions and displayed as a function of distance to the array (rank orders). For ipsilateral hemisphere, the decoding performance decreases as the distance to the electrode array increases (ρ=−0.649, P=0.003, n=18). For contralateral hemisphere, no correlation is observed (ρ=−0.074, P=0.770, n=18).

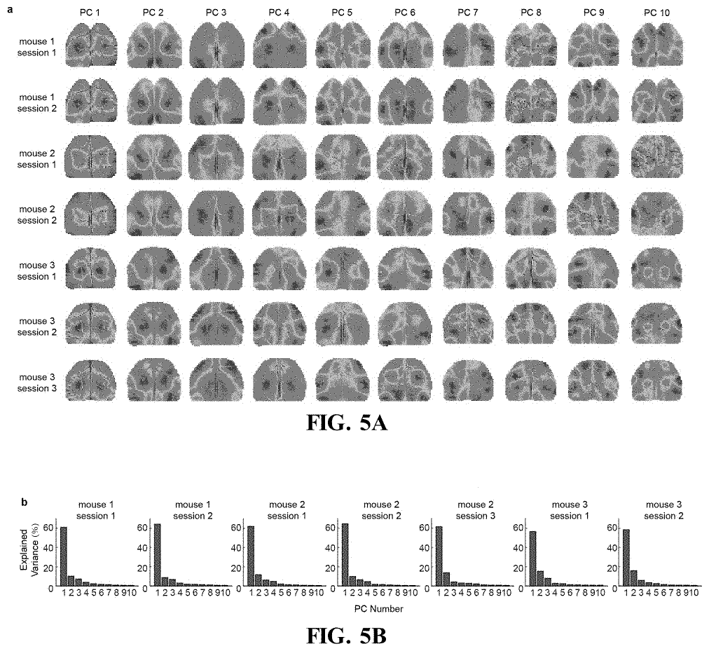

FIGS. 5A-5B show principal component analysis results for the cortical activity for all the animals. FIG. 5A shows identified principal components for individual recording sessions, showing different cortical co-activation patterns. FIG. 5B shows the proportion of variance explained by each principal component for individual recording sessions. For all the sessions, the top 10 principal components explained >92% variance in the data.

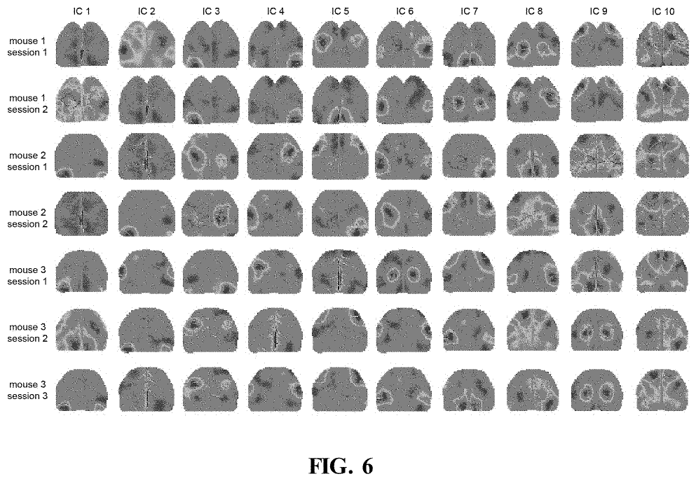

FIG. 6 shows independent components (ICs) for cortical activity for all the animals. Similar cortical functional modules and blood vessel activities are identified across different animals.

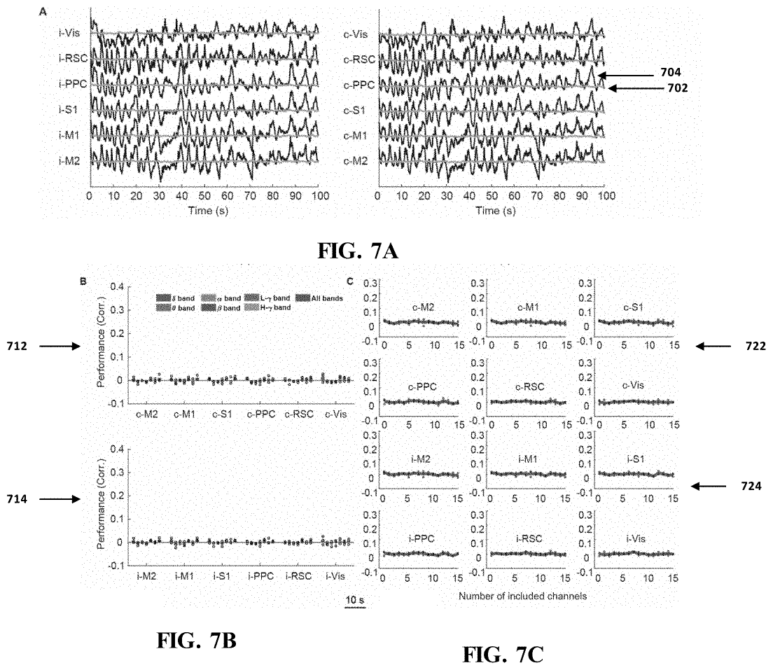

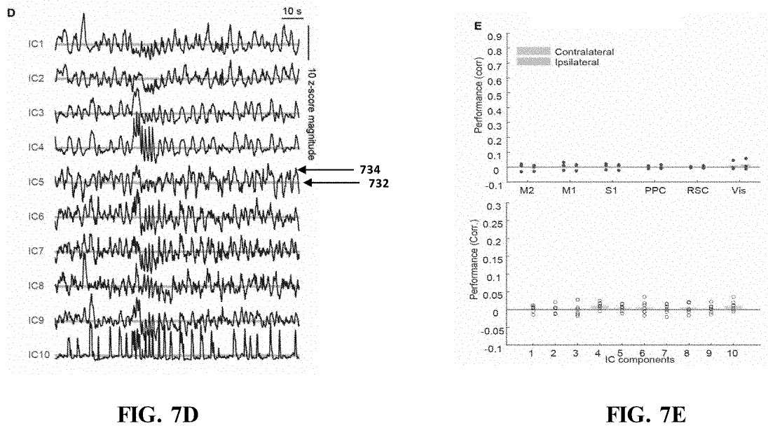

FIGS. 7A-7E show the decoding analysis using shuffled data. FIG. 7A shows the ground truth activity (704) and the decoded activity (702). FIG. 7B shows decoding performance evaluated for different cortical regions in the contralateral (712) and ipsilateral (714) hemispheres using shuffled data from different frequency bands. FIG. 7C shows Decoding performance for different cortical regions in the contralateral (722) and ipsilateral (724) hemispheres using shuffled data from all the frequency bands, but different numbers of recording channels. FIG. 7D shows decoded (732) and ground truth (734) weighting scores of the observed cortex-wide activity onto the 10 ICs shown in FIG. 4A using shuffled data. FIG. 7E shows decoding performance evaluated at pixel-level for all the cortical regions in the ipsilateral and contralateral hemispheres using shuffled data.

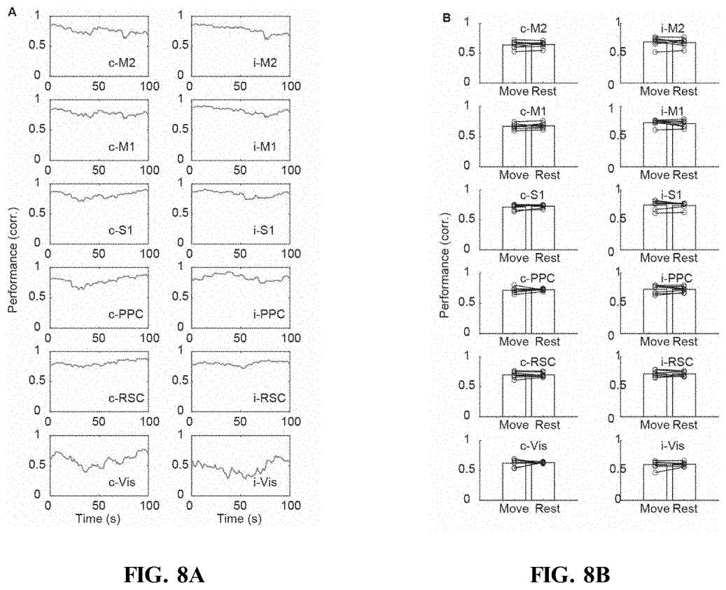

FIGS. 8A-8B show the stability of decoding performance and the effect of movement. FIG. 8A shows the decoding performance evaluated at 30 s sliding window for the data shown in FIG. 3A. FIG. 8B shows the decoding performance of individual cortical regions between movement and rest phases. Each dot marks the mean correlation evaluated by 10-fold cross-validation using the data recorded from one session.

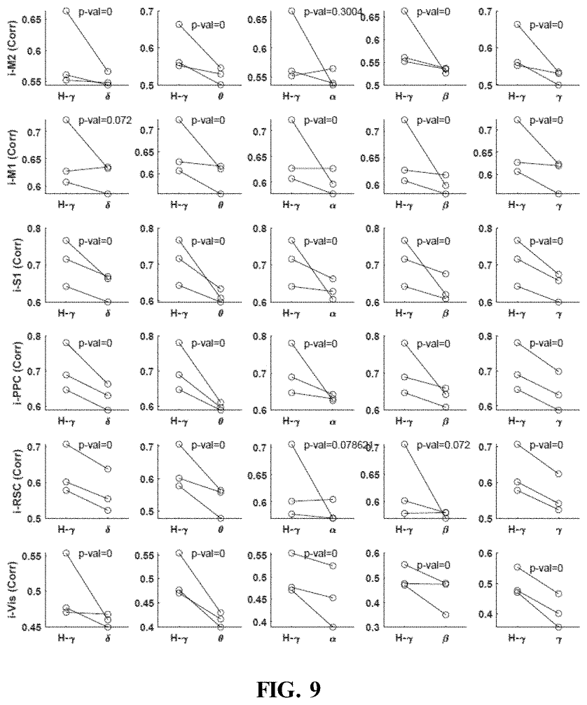

FIG. 9 shows comparison of the decoding performance using High-gamma power band vs. other frequency bands for ipsilateral cortical regions. For most cortical regions, the high-gamma power band gives significantly higher decoding performance than other frequency bands.

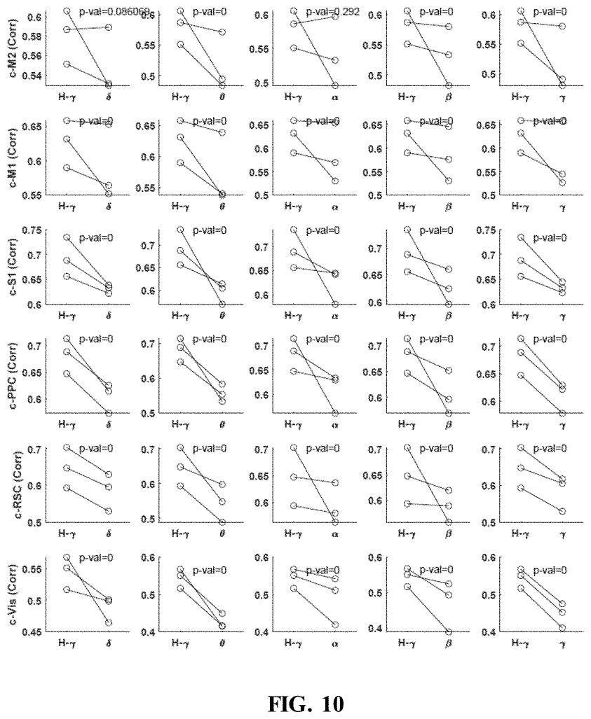

FIG. 10 shows comparison of the decoding performance using High-gamma power band vs. other frequency bands for contralateral cortical regions. For most cortical regions, the high-gamma power band gives significantly higher decoding performance than other frequency bands.

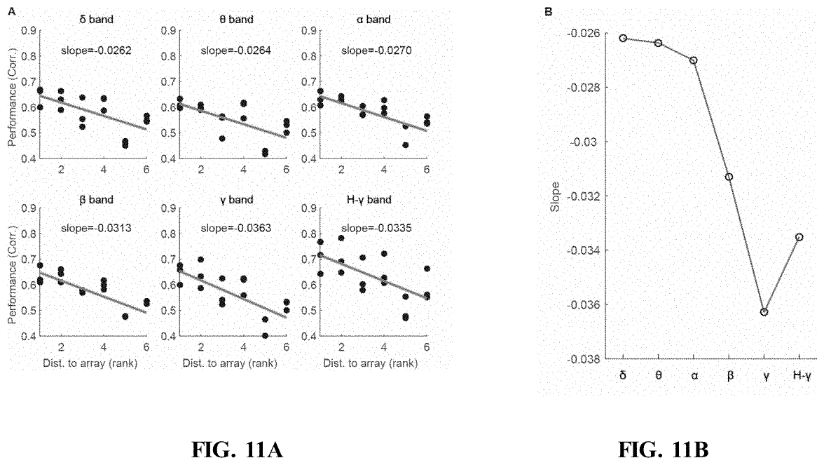

FIGS. 11A-11B show slope of the decoding performance for different frequency bands. FIG. 11A shows the decoding performance of ipsilateral cortices plotted against distance rank to the array for different frequency bands. FIG. 11B shows the slope of the decoding performance vs. distance for different frequency bands.

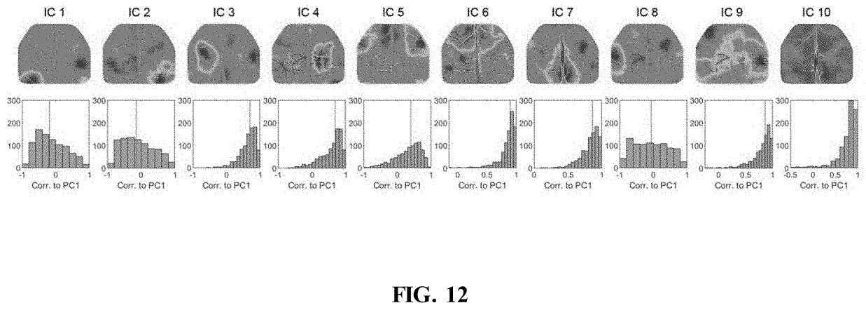

FIG. 12 shows the correlation between the activity of each ICs and the PC1. Top row shows the template for all the 10 ICs. Bottom row shows the histogram of the correlation between the activity of each IC and PC1 calculated for nonoverlapping 4 s segments during the recording. Note that the IC1, IC2 and IC8 have a median correlation close to zero, showing that their activities are not strongly correlated to PC1.

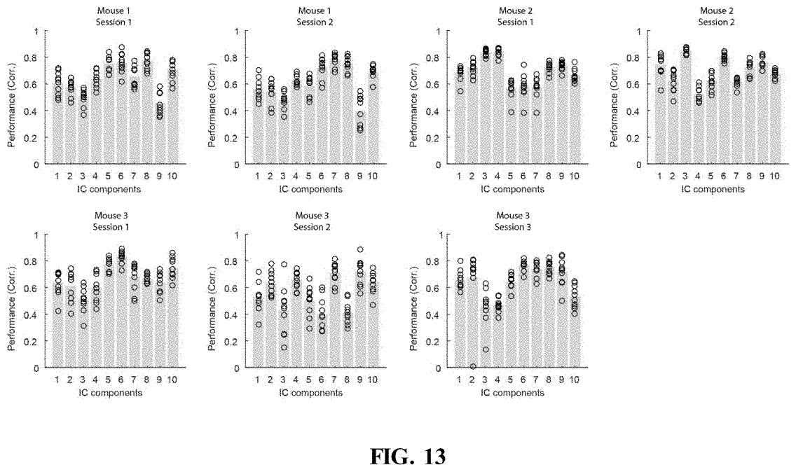

FIG. 13 shows decoding performance of the IC scores for all the sessions recorded for all the animals. Each dot marks one cross-validated correlation for one fold data.

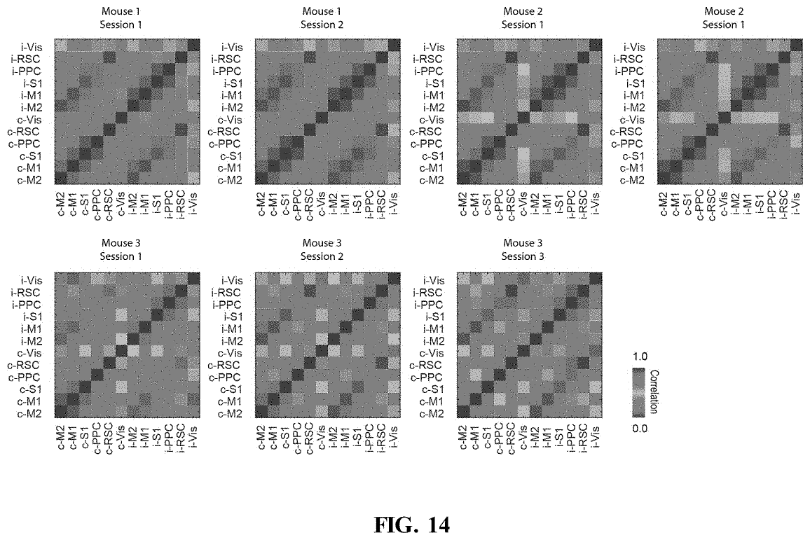

FIG. 14 shows correlation of ΔF/F activity from different cortical regions in all the mice. The same functional regions from both hemispheres often exhibit high correlation.

Electrical recordings of neural activity from brain surface have been widely employed in basic neuroscience research and clinical practice for investigations of neural circuit functions, brain—computer interfaces, and treatments for neurological disorders. Traditionally, these surface potentials have been believed to mainly reflect local neural activity. It is not known how informative the locally recorded surface potentials are for the neural activities across multiple cortical regions.

To investigate that, the disclosed technology can be implemented in some embodiments to perform simultaneous local electrical recording and wide-field calcium imaging in awake head-fixed mice. The disclosed technology can be implemented in some embodiments to use a recurrent neural network model to decode the calcium fluorescence activity of multiple cortical regions from local electrical recordings.

In some embodiments of the disclosed technology, the mean activity of different cortical regions may be decoded from locally recorded surface potentials. Also, each frequency band of surface potentials differentially encodes activities from multiple cortical regions so that including all the frequency bands in the decoding model gives the highest decoding performance. Despite the close spacing between recording channels, surface potentials from different channels provide complementary information about the large-scale cortical activity and the decoding performance continues to improve as more channels are included. In some embodiments, whole dorsal cortex activity at pixel-level can be decoded using locally recorded surface potentials.

These results show that the locally recorded surface potentials indeed contain rich information of the large-scale neural activities, which may be further demixed to recover the neural activity across individual cortical regions. In the future, the cross-modality inference approach based on some embodiments can be adapted to virtually reconstruct cortex-wide brain activity, greatly expanding the spatial reach of surface electrical recordings without increasing invasiveness. Furthermore, it may be used to facilitate imaging neural activity across the whole cortex in freely moving animals, without requirement of head-fixed microscopy configurations.

As an important tool for electrophysiological recordings, neural electrodes implanted on the brain surface have been instrumental in basic neuroscience research to study large-scale neural dynamics in various cognitive processes, such as sensorimotor processing as well as learning and memory. In clinical settings, neural recordings have been adopted as a standard tool to monitor the brain activity in epilepsy patients before surgery for detection and localization of epileptogenic zones initiating seizures and functional cortical mapping. Neural activity recorded from the brain surface exhibits rich information content about the collective neural activities reflecting the cognitive states and brain functions, which is leveraged for various types of brain—computer interfaces during the past decade. For example, surface potential recordings have been used for studying motor control, such as controlling a screen cursor or a prosthetic hand. They have also been used to decode the mood of epilepsy patients, paving the way for the future treatment of neuropsychiatric disorders. Recent advances have shown that electrical recordings from cortical surface combined with the recurrent neural networks can even enable speech synthesis, demonstrating superior performance compared to those achieved through traditional noninvasive methods.

For the interpretation of surface potentials in terms of their neural correlates, most research has focused on local neural activities. The high-gamma band has been found to correlate with the ionic currents induced by synchronous synaptic input to the underlying neuron population. Besides that, the dendritic calcium spikes in the superficial cortical layers also contribute to surface potentials. Recently, it has been reported that even the action potentials of superficial cortical neurons may be detected in surface recordings. Despite the predominant focus of relating the surface potentials to local neural activity, they may also correlate with the large-scale activity of multiple cortical regions. This may be achieved through the intrinsic correlations of the spontaneous activities among large-scale cortical networks due to the anatomical connectivity and the global modulation of neuromodulatory projections. However, this rich information content of surface potentials encoded for the large-scale cortical activity remains unexploited and little is known about how local surface potentials are correlated with the spontaneous neural activities of distributed large-scale cortical networks.

In some embodiments, the rich information content of the local neural potentials recorded from brain surface can be used to infer the cortex-wide brain activity. In some embodiments, optically transparent graphene microelectrodes implanted over the mouse somatosensory cortex and posterior parietal cortex (PPC) can be employed to perform simultaneous wide-field calcium imaging of the entire dorsal cortex during local neural recordings in awake mice. Multimodal datasets generated by these experiments are used to train a recurrent neural network model to learn the hidden spatiotemporal mapping between the local surface potentials and the cortex-wide brain activity detected by wide-field calcium imaging. In some embodiments, both the average spontaneous activity from multiple cortical regions and the pixel-level cortex-wide brain activity can be inferred from locally recorded surface potentials. The results obtained using some embodiments of the disclosed technology show that in addition to the changes of local neural activity, the spontaneous fluctuations of locally recorded surface potentials also reflect the collective variations of large-scale neural activities across the entire cortex.

Methods

Fabrication of Graphene Array

Electrode arrays are fabricated on 4″ silicon wafers spin coated with 20 nm thick PDMS. 50 μm thick PET (Mylar 48-02F-OC) is placed on the adhesive PDMS layer and used as the array substrate. 10 nm of chromium and 100 nm of gold are deposited onto the PET using a Denton 18 Sputtering System. The metal wires are patterned using photolithography and wet etching methods. Single-layer graphene is placed on the array area using a previously developed transfer process. The wafer is then soft baked for 5 min at 125° C. to better adhere graphene to the substrate. PMMA is removed via a 20 min acetone bath at room temperature then rinsed with isopropyl alcohol and DI water for ten 1 min cycles. The graphene channels are patterned using AZ1512/PMGI bilayer photolithography then oxygen plasma etched (Plasma Etch PE100). A four-step cleaning method is performed on the array consisting of an AZ NMP soak, remover PG soak, acetone soak, and ten-cycle isopropyl alcohol/DI water rinse. 8 nm thick SU-8 2005 is spun onto the wafer as an encapsulation layer and openings are created at the active electrical regions using photolithography. The array is then given a final ten-cycle isopropyl alcohol/DI water rinse to clean SU-8 residue and baked for 20 min at temperature progressing from 125° C. to 135° C.

Mice are group-housed in disposable plastic cages with standard bedding in a room with a reversed light cycle (12 h-12 h). Experiments are performed during the dark period. Both male and female healthy adult mice are used. Mice had no prior history of experimental procedures that may affect the results.

Surgery and Multimodal Experiments

Adult mice (6 weeks or older) are anesthetized with 1% and 2% isoflurane and injected with baytril (10 mg kg⁻¹) and buprenorphine (0.1 mg kg⁻¹) subcutaneously. A circular piece of scalp is removed to expose the skull. After cleaning the underlying bone using a surgical blade, a custom-built head-bar is implanted onto the exposed skull over the cerebellum (˜1 mm posterior to lambda) with cyanoacrylate glue and cemented with dental acrylic (Lang Dental). Two stainless-steel wires (791 900, A-M Systems) are implanted into the cerebellum as ground/reference. A craniotomy (˜7 mm×8 mm) is made to remove most of the dorsal skull and the graphene array is placed on the surface of one hemisphere, covering somatosensory cortex (S1) and PPC. The exposed cortex and the array are covered with a custom-made curved glass window, which is further secured with an adhesive (e.g., Vetbond (3 M)), cyanoacrylate glue and dental acrylic. Animals are fully awake before recordings. During recording, animals are head-fixed under the microscope, free to run or move their body, and not engaged in task.

The wide-field calcium imaging is performed using a commercial fluorescence microscope (e.g., Axio Zoom V16, Zeiss, objective lens (1×, 0.25 NA)) and a CMOS camera (e.g., ORCA-Flash4.0 V2, Hamamatsu) through the curved glass window as previously described. The light source for wide-field calcium imaging is used (e.g., HXP 200 C (Zeiss)). The filter set for imaging GCaMP signals is commercially installed in the microscope. It consists of a bandpass filter for the excitation light (485±17 nm), a beamsplitter (500 nm), and a tunable bandpass filter centered at 520 nm for the emission light. Images are acquired using an imaging application (e.g., HCImage Live (Hamamatsu)) at 29.98 Hz, 512×512 pixels (field of view: 8.5 mm×8.5 mm, binning: 4, 16 bit).

The microelectrode array is connected to a custom-made connector board through a ZIF connector. The surface potential data is sampled with electrophysiology amplifier (e.g., Intan RHD2132) and recorded using a recording system (e.g., IntanRHD2000). The sampling frequency is 10 kHz. To synchronize the electrical recording with the calcium imaging, a trigger signal (TTL), a 2 V pulse of 1 s, can be used to trigger the start of the calcium imaging. Meanwhile, this trigger signal is also sent to the ADC of the recording system. During the data processing stage, the onset of the pulse can be detected and the imaging data and electrical data can be aligned to that time point. Three mice are recorded, each having two to three recording sessions. The length for each recording session is 1 h.

ΔF/F Processing

To obtain the ΔF/F time series from the wide-field calcium imaging data, the 512×512 pixel images can be first down-sampled to smaller images of 128×128 pixels. In some implementations, ΔF/F indicates the change in fluorescence intensity. For each pixel, a dynamic fluorescence (F) baseline for a given time point can be defined as the 10th percentile value over 180 s around it. For the beginning and ending of each imaging block, the following and preceding 90 s window is used to determine the baseline, respectively. An 8th order 6 Hz Butterworth low-pass filter is applied to the ΔF/F activity of each pixel to remove the high frequency noise and hemodynamic contamination from heartbeat. The activity of each cortical region is obtained by averaging over the ΔF/F signals from all the pixels within the same cortical regions defined by the Allen Brain Atlas.

Surface Recording Data Processing

The raw surface recording data is first passed through notch filters to eliminate the 60 Hz powerline contaminations and their higher harmonics at 120 Hz and 180 Hz. The signals are further filtered with multiple 6th order Butterworth band-pass filters designed for different frequency bands (δ: 1-4 Hz, θ: 4-7 Hz, α: 8-15 Hz, β: 15-30 Hz, γ: 31-59 Hz, H_{γ}: 61-200 Hz). The resulting signals are squared and smoothed by a Gaussian function with 100 ms time window to obtain an estimate of the instantaneous power. To prepare the input data for the decoding neural network, the power traces at different frequency bands are down-sampled to 29.98 Hz by interpolation to match the sampling rate of calcium imaging data. To suppress the potential artifacts in the recording signal, at each frequency band, the power traces are clipped with a threshold of 95 percentile.

Neural Network Models

The neural network model consists of a sequential stacking of a linear hidden layer, one bidirectional LSTM layer and a linear readout layer. The 1st linear layer is followed by batch normalization, ReLU activation, and dropout with a probability of 0.3. The LSTM layer is followed by batch normalization. The multichannel power at different frequency bands can be used as inputs to the network. To decode the neural activity at each time step t, the power segments between [t−1.5 s, t+1.5 s] is used (90 time steps in total). The 1st linear layer had 16 neurons and the bidirectional LSTM had eight hidden neurons. The same neural network model is used for the two decoding tasks except that the number of neurons in the final output layer differs based on the targeting output. To decode the ΔF/F activity of 12 cortical regions simultaneously, the output neuron number is set to 12. To decode the cortex-wide brain activity, the output neuron number is set to ten to generate the scores for the ten ICs. Assuming using six frequency bands from 16 recording channels, setting sequence length of LSTM layer to 90, and setting batch size to 128, the input and output size for each layer of the model is shown in Table 1. In some embodiments, the last two dimensions of the LSTM output are flattened to make it 128×1440 before feeding it to the last linear layer.

In some embodiments, the neural network model can be implemented in a machine learning framework (e.g., Pytorch). The model parameters are trained through Adam optimizer with learning rate=1×10⁻⁴, beta1=0.9, beta2=0.999, epsilon=1×10⁻⁸. The batch size is 128 and the training usually converged within ˜30 epochs. For both tasks, the mean squared error is chosen as the loss function. The disclosed technology can be implemented in some embodiments to perform ten-fold cross-validation where each 1 h recording session is chunked into ten segments, each lasting for 6 min. The neural network model is trained on 9/10 of the data segments and tested on a different held-out segment that is unseen during the training. To evaluate the model performance, correlation between the decoded and ground truth data for each held-out set is averaged. For each 1 h recording session, a new network model is trained and tested. Then, for each mouse, the correlation is further averaged across the recording sessions to give the performance for that mouse.

TABLE 1
The size for input and output tensors of each layer.

| | Input size | Output size |
|---|---|---|
| First linear layer | 128 × 90 × 96 | 128 × 90 × 16 |
| Bi-LSTM layer | 128 × 90 × 16 | 128 × 90 × 16 |
| Last linear layer | 128 × 1440 | 128 × 12 or 128 × 10 |

Statistical Tests

All statistical analyses are performed in MATLAB. Statistical tests are two-tailed and significance is defined by alpha pre-set to 0.05. All the statistical tests are described in the figure legends. Multiple comparisons are corrected for by Benjamini-Hochber corrections.

Multimodal Recordings of Cortical Activity

Cortical recordings in both clinical applications and neuroscience studies use conventional metal-based neural electrode arrays. However, these opaque neural electrodes are not suitable for multimodal recordings combined with optical imaging since they will block the field of view and generate light-induced artifacts under optical imaging. Compared to conventional neural electrode arrays, graphene-based surface arrays are optically transparent and free from light-induced artifacts, both of which are key to the simultaneous electrical recordings and optical imaging of cortical activity. Wide-field calcium imaging is an optical imaging technique that can provide simultaneous monitoring of large-scale cortical activity and has been used to study the dynamics of multiple cortical regions and their coordination during behavior. Compared to functional magnetic resonance imaging (fMRI) that also offers large spatial coverage, the wide-field calcium imaging provides better spatiotemporal resolution and higher signal-to-noise ratio. It has been shown that wide-field calcium signals mainly reflect local neural activity. Therefore, the multimodal experiments combining electrical recordings based on graphene arrays and the wide-field calcium imaging generate unique datasets that are ideal for investigating the mapping from local neural signals to large-scale cortical activity.

The disclosed technology can be implemented in some embodiments to fabricate transparent graphene arrays on 50 μm thick flexible polyethylene terephthalate (PET) substrates. 10 nm of chromium and 100 nm of gold are deposited onto the PET and the metal wires are patterned using photolithography and wet etching methods. The graphene layer is transferred and patterned with photolithography and oxygen plasma etching to form electrode contacts. Finally, 8 μm thick SU-8 is used as an encapsulation layer and openings are created at the active electrical regions using photolithography. The graphene array has 16 recording channels, each of size 100×100 μm. The spacing between adjacent channels is 500 μm. The graphene array is implanted unilaterally over the somatosensory cortex (S1) and PPC of the mice to perform the simultaneous electrical recordings and wide-field calcium imaging (FIG. 1A). The disclosed technology can be implemented in some embodiments to perform multimodal recordings of spontaneous neural activity in awake mice during either quiet resting state or actively running or moving. An example wide-field image obtained during the experiment is shown in FIG. 1B. Note that the cortical activity under the array may still be observed due to the transparency of the graphene electrode. Based on Allen brain atlas, the brain is parcellated into 12 different ipsilateral (the hemisphere with array implanted) and contralateral cortical regions (FIG. 1C), including the primary and secondary motor cortices (M1, M2), the somatosensory cortex (S1), the PPC, the retrosplenial cortex (RSC), and the visual cortex (Vis). Representative spontaneous cortical activity recorded during the experiment is shown in FIG. 1D. Dynamical changes of large-scale cortical activity, involving co-activations of multiple cortical regions, are observed. In the simultaneous multi-channel neural recordings, differences are also observed in power traces from different channels at multiple frequency bands during the spontaneous cortical activity (FIG. 1E). Compared with the fluorescence activity, the neural potential signal has a much higher temporal resolution and richer frequency components.

Cortical Activity Decoder Design

The disclosed technology can be implemented in some embodiments to infer the cortex-wide brain activity by using the locally recorded surface potentials. In some embodiment, two decoding tasks, (1) the decoding of the average activity from individual cortical regions and (2) the decoding of pixel-level cortex-wide brain activity, can be performed to infer the cortex-wide brain activity. In some embodiments, a compact neural network model consisting of a linear hidden layer, a one-layer LSTM network, and a linear readout layer (FIG. 2), can be used. In both tasks, the signal power traces of multiple frequency bands recorded from different recording channels are used as inputs to the neural network. In the 1st task, the neurons in the output layer of the neural network directly generate the activity of all the cortical regions simultaneously. In the 2nd task, principal component analysis (PCA) is first performed on the cortical activity to remove the noise and reduce the dimensionality of the data. Across all the mice, the top ten PCs explain >92% variance in the data (FIGS. 5A-5B). Then based on the PCA results, spatial independent component analysis (ICA) is further performed to obtain the independent components (ICs) and their weighting scores for the data at each time frame. In all the three mice, the identified ICs reflect different functional modules and hemodynamic signals on blood vessels (FIG. 6) and provide a set of functionally meaningful basis for the decomposition of the large-scale cortical activity. The output layer of the neural network directly generates the estimated weighting scores of individual ICs, which are further used to reconstruct the cortex-wide brain activity at each time frame with pixel-level spatial resolution (FIG. 2).

Decoding of Activity for Individual Cortical Regions

Based on the multimodal data collected during the animal experiment and the above designed decoder network model, the mean activity of both the ipsilateral and contralateral cortical regions can be decoded using the power of six frequency bands from all recording channels. An example of decoded and ground truth (ΔF/F from wide-field calcium imaging) cortical activity from one held-out set is shown in FIG. 3A. The decoding performances for S1, PPC, and RSC regions closely resemble the ground truth cortical activity, while the decoding performances for M1, M2, and Vis are lower, possibly due to their increasing distances to the recording electrode array. In some embodiments, the same decoding analysis can be performed using shuffled data. The results show decoding performance close to zero (FIG. 7A). The stability of the decoding performance is evaluated across time using a 30 s sliding window. The results show that the decoding performance fluctuates from time to time but remains stable in the longer time intervals (FIG. 8A). In addition, the decoding performance of individual cortical regions during rest and movement intervals can be compared, and similar decoding performance between rest and movement phases can be found (FIG. 8B). Therefore, the fluctuations of the decoding performance across time are not due to animal movements.

To further evaluate how informative different frequency bands are for the decoding of the activity from different cortical regions, the signal power from different frequency bands of all channels can be used as inputs and ten-fold cross-validation can be performed to evaluate the decoding performance of the neural network model. In some embodiments, even though all the frequency bands are informative of the activities in different cortical regions, the high gamma power band gives the highest decoding performance for all the cortical regions compared to other frequency bands (FIGS. 9 and 10). However, across all the cortical regions, using all the frequency bands yields the best decoding performance compared to using any single frequency band (FIG. 3(b)), implying that different frequency bands provide complementary information about the activity in multiple cortical regions. Decoding with the shuffled data gives performance close to zero for all the frequency bands (FIG. 7B). For the ipsilateral cortical regions, a negative correlation between their decoding performance and their distance ranks to the recording array can be found. However, for the contralateral cortical regions, no significant correlation is observed (FIG. 3C). When comparing the decoding results of the activity from ipsilateral cortical regions using different frequency bands, higher frequency bands tend to have a steeper slope for the decoding performance vs. distance to the recording array (FIGS. 11A-11B).

Besides the frequency bands, different recording channels encode nonredundant information for decoding the activity of different cortical regions. Therefore, the decoding performance of the neural network model can be evaluated using all six frequency bands from different numbers of channels. Specifically, ten-fold cross-validation can be performed on the neural network multiple times and each time the signal power of all frequency bands is sequentially added from one random channel until all the channels are included. As shown in FIG. 3D, for all the cortical regions, increasing the number of channels significantly improves the decoding performance, suggesting that recording channels of local neural potentials provide nonredundant information about the activity from multiple cortical regions. On the other hand, decoding with the shuffled data gives performance close to zero for different number of included channels (FIG. 7C).

Decoding of Pixel-Wise Activity Across Cortex

Given that the local neural signals encode average activity from individual cortical regions, which may be recovered by the neural network model using multi-channel signal power of different frequency bands, the pixel-level activity across the whole dorsal cortex may also be decoded using locally recorded neural signals. The same neural network model for decoding the average activity in different cortical regions is then employed to simultaneously decode the ten IC scores at each time frame. The power traces of all the six frequency bands from all the recording channels are used as inputs to the neural network. An example of the decoded and ground truth scores for the ten ICs from one held-out set is shown in FIG. 4B. The decoding result using shuffled data is shown in FIG. 7D. Based on the decoded IC scores and the IC modules (FIG. 4A), the pixel-level cortex-wide activity at each time frame may be reconstructed. Examples of the reconstructed pixel-level cortex-wide activity during four representative time intervals are shown in FIG. 4C. The reconstructed cortex-wide activity captured various patterns of cortical activations in ground truth, including both the synchronous and asynchronous activations among different cortical regions. These diverse activation patterns cannot be explained solely by PC1 (see FIG. 4C). To further quantify this observation, the correlation between the ground truth activity of each ICs and the PC1 can be computed. The median correlations between IC1, IC2 and IC8 to PC1 are close to zero, showing that their activities are not strongly correlated to PC1 (FIG. 12). These results suggest that the model does not merely predict dominant activity patterns showing activation around S1 and RSC. In some embodiments, all the ten IC scores may be decoded using the locally recorded neural signals (FIG. 4D and FIG. 13). In some embodiments, the pixel-level cortex-wide activity may be reconstructed for all the recording. This reveals that the cortical activations of distinct functional modules indeed induce different responses in local cortical electrical signals, which may be in turn used to recover the diverse cortex-wide activity patterns. In addition to cortical activity, in all the mice, one or two ICs showing the hemodynamic activity (FIG. 6) can be observed. The decoding results also show that these hemodynamic activities may be decoded from the neural recordings, which is mainly due to the fact that hemodynamic activity and the neural activity are often correlated. The disclosed technology can be implemented in some embodiments to examine the pixel-level correlations between the decoded and ground truth activities imaged using wide-field imaging in individual cortical regions. In some embodiments, high correlations between the decoded and the ground truth data for all cortical regions (FIG. 4E) and close-to-zero correlations using shuffled data can be observed (FIG. 7E). The activities of cortical regions closer to the array are better decoded than those of the cortical regions far away from the array. Consistent with the decoding of mean activity in each cortical region, the pixel-wise correlation decreases as the distance rank to the surface array increases for the ipsilateral cortical regions, whereas for the contralateral cortical regions no such correlation exists (FIG. 4F).

The disclosed technology can be implemented in some embodiments to perform multimodal recordings of local neural potentials and wide-field calcium imaging in awake mice and developed a recurrent neural network model to decode the large-scale spontaneous cortical activity from the locally recorded multi-channel electrical signals. Both the averaged and the pixel-level activity across the entire dorsal cortex may be decoded, and the best decoding performance is achieved using all frequency bands of recorded neural potentials. These results suggest that even though the cortical electrical recording is a complex signal contributed by various mechanisms at multiple spatial scales, the responses in individual frequency bands across multiple recording channels still provide important discriminative information about the activity of different cortical regions. By developing a decoder model, the mixed information in the electrical signal responses may be used to recover the simultaneously recorded cortex-wide brain activity.

The cortical potentials have long been believed to mainly detect local neural activities that are within a sensing distance between 500 μm to 1-3 mm, depending on the size of the electrode as well as the spatial correlation pattern of neural activity. Consistent with this claim, for the decoding of mean activity from individual cortical regions, there can be a decreasing decoding performance for the ipsilateral cortical regions located ˜1.5-3 mm from the array. Interestingly, for the contralateral cortical regions, the decoding is still possible even though their activities are unlikely to be directly detected by the neural electrodes. In some embodiments, the successful decoding of contralateral cortical regions is mainly due to the fact that the spontaneous activities of same functional cortical regions in both hemispheres are often correlated (FIG. 14). Such correlated activity may arise from the anatomical connectivity and further orchestrated by neuromodulatory projections.

In some embodiments, the decoding results for the activity of individual cortical regions show that even with single recording channel, the decoding is possible (mean correlation performance between 0.35 and 0.65 for different cortical regions). By including more channels, an increase in decoding performance is initially observed, but the performance starts to saturate after the inclusion of ten recording channels (mean correlation performance between 0.6 and 0.75 for different cortical regions). This can be mainly because of the fact that the neural potentials in adjacent channels are partially correlated due to the volume conduction in the brain tissue. It has been shown that the correlation between neural potentials from adjacent channels at different frequency bands decreases as the distance increases. Even though the cross-channel correlation at high frequency bands is lower than that at low frequency bands, it does not go below chance level even with a distance of ˜1.5 mm. However, the results empirically confirm that even though the neural potentials from adjacent channels are partially correlated, they still differentially encode information about the cortical activities to some extent so that sequentially including more recording channels tends to increase the decoding performance. However, beyond a certain threshold adding more channels does not further increase the decoding performance.

For the decoding of cortex-wide brain activity, instead of attempting to directly reconstruct the activity of individual pixels, the disclosed technology can be implemented in some embodiments to perform PCA followed by spatial ICA on the cortical activity and later to decode IC scores to recover the cortex-wide activity at pixel level. The adoption of this approach is based on both scientific and computational considerations. First, the PCA effectively reduced the spatial dimensions, while preserving a large proportion of variance in cortical activity. Since the activity of each single pixel is noisy, performing PCA reduced the noise, leading to a more reliable estimate of the true activity. Second, choosing the IC scores as network outputs greatly reduced the parameters in the output layer of the neural network model, prevented overfitting, and speeded up model training. Finally, the spontaneous cortex-wide brain activity is decomposed into a set of local and spatially organized cortical activation patterns based on neural activity, generating a biologically meaningful decomposition that matches the brain dynamics. This decomposition provides a good demixing of cortex-wide brain activity and enables a tractable mapping from cortical neural responses, which can be learned by the decoding network model. Taken together, these results reveal that the activation of different cortical functional modules are associated with distinct components in local neural activity. By exploiting the mapping between the two modalities, the decoding of cortex-wide brain activity is possible from locally recorded neural signals.

The disclosed technology can be implemented in some embodiments to provide a neural network model to show that both the mean activity of different cortical regions and the pixel-level cortex-wide neural activity can be decoded using locally recorded surface potentials. These findings demonstrated that the locally recorded neural potentials indeed contain rich information for large-scale neural activity and the surface potential responses in different frequency bands and different recording channels provide distinct information about the large-scale neural activity.

The disclosed technology can be implemented in some embodiments to obtain virtual imaging of cortex-wide brain activity from locally recorded ECoG signals.

From basic neuroscience research to clinical applications, electrocorticography (ECoG) can be used to record electrical potentials from brain surface to localize seizure onset zones for presurgical planning and to map out functional cortical regions. However, invasive implantation process involving removal of the skull significantly limits the spatial coverage of ECoG arrays and prevents recording from cortical networks across large areas. The disclosed technology can be implemented in some embodiments to demonstrate virtual imaging of cortex-wide activity using locally recorded ECoG potentials and a recurrent neural network model trained with multimodal dataset generated by simultaneous ECoG recordings and wide-field calcium imaging. The disclosed technology can be implemented in some embodiments to demonstrate that both the average activity of different cortical regions and the pixel-level cortex-wide activity can be decoded virtually reconstructed using local ECoG recordings. The disclosed technology can be implemented in some embodiments to provide a new approach for cross-modality inference of cortex-wide brain activity, enhancing the ability of ECoG recordings to monitor large-scale cortical activity without increasing invasiveness.

As a widely used electrophysiological tool, ECoG senses the electrical activity of the cortex by placing the electrodes directly over the brain surface. Compared with the conventional noninvasive methods, such as electroencephalography (EEG), ECoG provides a broader frequency range and higher temporal and spatial resolution (˜1 ms, ˜1 mm) with spatial coverage depending on the size of the craniotomy. It has been suggested that ECoG signals contain rich information about the collective neural activities, the cognitive states and the brain functions. In basic neuroscience research, ECoG has been used to study the large-scale neural dynamics, sensorimotor processing as well as learning and memory. Different ECoG frequency bands have been identified to correlate with various behavioral states and cognitive processes. The slow-wave band (<1 Hz) and delta band activity (1-4 Hz) has been signatures of the non-rapid eye movement sleep, reflecting the synchronous hyperpolarized and depolarized states of cortical neurons. The theta band activity (4-8 Hz) has been suggested to coordinate among different cortical regions. The beta band activity (15-30 Hz) decreases during the movement initiation and increases after behavioral stopping. The gamma band activity (30-200 Hz) mainly reflects firing of local neuron population and increases under visual stimulus and may be modulated by theta band or alpha band waves. In clinical settings, ECoG has been adopted as a standard tool to monitor the brain activity of epilepsy patients before surgery for detection and localization of epileptogenic zones initiating seizures and functional cortical mapping. During the past decade, ECoG has emerged as a promising technique for various types of brain-computer interfaces. The ECoG signals have been used for motor tasks, such as decoding the grasping types, controlling a screen cursor or a prosthetic hand. ECoG have also been used to decode the mood of epilepsy patients, paving the way for the future treatment of neuropsychiatric disorders. Recent advances have shown that ECoG recordings combined with the recurrent neural networks can even enable speech synthesis, demonstrating superior performance compared to those achieved through traditional noninvasive methods.

For the interpretation of ECoG signals in terms of their neural correlates, most research has focused on local neural activities. The ECoG high-gamma power has been found to correlate with the ionic currents induced by synchronous synaptic input to the underlying neuron population. Besides that, the dendritic calcium spikes in the superficial cortical layers also contribute to surface potentials. Recently, it has been reported that even the action potentials of superficial cortical neurons may be detected in ECoG recordings. Despite this predominant focus of relating the ECoG signals to local neural activity, the ECoG signals may also correlate with or indirectly influenced by the activity of other cortical regions. This may be achieved through the intrinsic correlations of the spontaneous activities among large-scale cortical networks due to the cortico-cortical connectivity and the neuromodulatory projections. However, this rich information content of recorded ECoG signals encoded for the large-scale cortical activity remains largely unexploited compared to that for the local neural activities. Investigating functions of spatially distributed cortical networks and functional interactions between different cortical regions would require to implant multiple ECoG electrodes with large spatial coverage. However, this would significantly increase the invasiveness of the surgery, substantially increasing the risk of infection. A less invasive method that may provide similar information and resolution to ECoG across large areas of the brain would be transformative for both clinical and neuroscience applications.

In some embodiments, the large-scale cortex-wide neural activity can be inferred from locally recorded ECoG signals using multimodal recordings enabled by transparent ECoG technology and recurrent neural networks. Optically transparent graphene microelectrode arrays allow simultaneous wide-field calcium imaging of the entire dorsal cortex during ECoG recordings. Optically transparent graphene microelectrode arrays are implanted over the mouse somatosensory cortex and simultaneous ECoG recordings and wide-field calcium imaging of the dorsal cortex are performed in awake mice. Multimodal datasets generated by these experiments are used to train a recurrent neural network model towards learning the hidden spatiotemporal mapping between the ECoG signals and the cortex-wide neural activity detected by calcium imaging. The disclosed technology can be implemented in some embodiments to “virtually” image spontaneous activity from multiple cortical regions and infer the cortex-wide brain activity from local ECoG potentials with pixel-level spatial resolution.

The disclosed technology can be implemented in some embodiments to fabricate transparent graphene arrays on flexible and transparent polyethylene terephthalate (PET) substrates (see Methods for details). 50 μm thick transparent PET substrates are used. 10 nm of chromium and 100 nm of gold are deposited onto the PET and the metal wires are patterned using photolithography and wet etching methods. The graphene layer is transferred and patterned with photolithography and oxygen plasma etching to form electrode contacts. Finally, 8 μm-thick SU-8 is used as an encapsulation layer and openings are created at the active electrical regions using photolithography. The graphene array has 16 recording channels, each of size 100×100 The spacing between adjacent channels is 500 μm. The graphene array is implanted unilaterally over the somatosensory cortex (S1) of the mice to perform the simultaneous electrical recordings and wide-field calcium imaging (FIG. 1A). An example wide-field image obtained during the experiment is shown in FIG. 1B. Note that the cortical activity under the array may still be observed due to the transparency of the graphene electrode. Based on Allen brain atlas, the brain is parcellated into 12 different ipsilateral (the hemisphere with array implanted) and contralateral cortical regions (FIG. 1C), including the primary and secondary motor cortices (M1, M2), the somatosensory cortex (S1), the posterior parietal cortex (PPC), the retrosplenial cortex (RSC), and the visual cortex (Vis). Representative spontaneous cortical activity recorded during the experiment is shown in FIG. 1d. In some embodiments, there can be dynamical changes of large-scale cortical activity that involves the co-activation of multiple cortical regions. In the simultaneous multi-channel ECoG recordings, there can be differences in power traces from different ECoG channels at multiple frequency bands during the spontaneous cortical activity (FIG. 1E). Compared with the fluorescence activity, the ECoG signal has a much higher temporal resolution and richer frequency components.

Cortical Activity Decoder Design

In some embodiments of the disclosed technology, the overall activity of individual anatomical cortical regions may be decoded from the locally recorded ECoG signals. This is crucial for virtual imaging of large-scale cortical network dynamics and communication between different cortical regions. It is also important to further investigate the different neural activities within each anatomical cortical region with a much finer spatial resolution, which may significantly expand the clinical and research applications of virtual imaging. To that end, two decoding tasks are performed, namely the decoding of ΔF/F activity from individual cortical regions and the decoding of pixel-level cortex-wide brain activity. To achieve these goals, the disclosed technology can be implemented in some embodiments to provide a compact neural network model consisting of a linear hidden layer, a one-layer LSTM network, and a linear readout layer (FIG. 2, See Methods for details). In both tasks, the ECoG power traces of multiple frequency bands recorded from different ECoG channels are used as inputs to the neural network. In the first task, the neurons in the output layer of the neural network directly generate the activity of all the cortical regions simultaneously. In the second task, principal component analysis (PCA) is first performed on the cortical activity to suppress the noise and reduce the dimensionality of the data. Across all the mice, the top 10 PCs explain >92% variance in the data (FIGS. 5A-5B). Then based on the PCA results, spatial independent component analysis (ICA) is further performed to obtain the independent components (ICs) and their weighting scores for the data at each time frame. In all the three mice, the identified ICs reflect different functional modules and blood vessel activity (FIG. 6) and provide a set of functionally meaningful basis for the decomposition of the large-scale cortical activity. The output layer of the neural network directly generates the estimated weighting scores of individual ICs, which are further used to reconstruct the cortex-wide brain activity at each time frame with pixel-level spatial resolution.

Virtual Imaging of Activity for Individual Cortical Areas

Based on the multimodal data collected during the animal experiment and the above designed decoder network model, the activity of both the ipsilateral and contralateral cortical regions are virtually imaged by decoding the mean cortical activity from the ECoG power of six frequency bands from all ECoG channels. An example of decoded and ground truth cortical activity from one held-out set is shown in FIG. 3A. To further evaluate how informative different frequency bands are for the decoding of the activity from different cortical regions, the ECoG power from different frequency bands of all ECoG channels can be used as inputs and performed 10-fold cross-validation to evaluate the decoding performance of the neural network model. In some embodiments, even though all the frequency bands are informative of the activities in different cortical regions, the high gamma power band gives the highest decoding performance for all the cortical regions compared to other ECoG frequency bands. However, across all the cortical areas, using all of the ECoG frequency bands yields the best decoding performance compared to using any single frequency band (FIG. 3B), implying that different ECoG frequency bands provide complementary information about the activity in multiple cortical regions. For the ipsilateral cortical regions, in some embodiments, a negative correlation between their decoding performance and their distances to the recording array. However, for the contralateral cortical areas, no significant correlation is observed (FIG. 3C).

In some embodiments, besides the ECoG frequency bands, different ECoG channels encode nonredundant information for decoding the activity of different cortical regions. Therefore, the decoding performance of the neural network model using all six frequency bands from different number of ECoG channels is evaluated. Specifically, 10-fold cross-validation is performed on the neural network multiple times and each time the ECoG power of all frequency bands can be sequentially added from one random ECoG channel until all the ECoG channels are included. As shown in FIG. 3D, for all the cortical areas, increasing the number of ECoG channels significantly improves the decoding performance, suggesting that ECoG channels provide nonredundant information about the activity from multiple cortical regions.

Virtual Imaging of Pixel-Wise Activity Across Cortex

Given that the local ECoG signals encode activity from individual cortical regions, which may be recovered by the neural network model using multi-channel ECoG power of different frequency bands, the pixel-level activity across the whole dorsal cortex may also be virtually reconstructed using locally recorded ECoG signals. The same neural network model for decoding the average activity in different cortical regions is then employed to simultaneously decode the ten IC scores at each time frame. The ECoG power traces of all the six frequency bands from all the recording channels are used as inputs to the neural network. An example of the decoded and ground truth scores for the ten ICs from one held-out set is shown in FIG. 4B. Based on the decoded IC scores and the IC modules (FIG. 4A), the pixel-level cortex-wide activity at each time frame may be reconstructed. Examples of the reconstructed pixel-level cortex-wide activity during 4 representative time intervals are shown in FIG. 4C. In some embodiments, all the ten IC scores may be decoded using the locally recorded ECoG signals (FIG. 4D, FIGS. 7A-7E) and the pixel-level cortex-wide activity may be reconstructed for all the recording sessions. This reveals that the cortical activations of distinct functional modules indeed induce different responses in ECoG signals, which may be in turn used to recover the diverse cortical activity. In addition to cortical activity, in all the mice, one or two ICs can show the hemodynamic activity (FIG. 6). The decoding results based on some embodiments also show that the hemodynamic activity may be decoded from the ECoG signals, which is mainly due to the fact that hemodynamic activity and the neural activity are often correlated. Next, the pixel-level correlations between the decoded and ground truth activities in different cortical areas are examined. The result shows high correlations like the previous decoding task for average activity of individual cortical regions (FIG. 4E, compare to FIG. 3B), The activities of cortical regions closer to the array are better decoded than those of the cortical areas far away from the array. For the ipsilateral cortical areas, the pixel-wise correlation decreases as the distance to the ECoG array increases, whereas for the contralateral cortical areas no such correlation exists (FIG. 4F).

In some embodiments of the disclosed technology, multimodal recordings of ECoG signals and wide-field calcium imaging in awake mice can be performed, and a recurrent neural network model can be used to decode the large-scale spontaneous cortical activity from the locally recorded multi-channel ECoG signals. Both the averaged and the pixel-level activity across large cortical areas may be recovered. These results demonstrate that even though ECoG is a complex signal contributed by various different mechanisms at multiple spatial scales, the responses in individual frequency bands across multiple ECoG channels still provide discriminative information about the activity of different cortical regions. By developing a decoder model, the mixed information in the ECoG responses may be used to recover the simultaneously recorded cortex-wide brain activity.

The ECoG signals have long been believed to mainly detect local neural activities that are within a sensing distance between 500 μm to 1-3 mm, depending on the size of the electrode as well as the spatial correlation pattern of neural activity. Consistent with this claim, for the decoding of mean activity from individual cortical areas, there can be a decreasing decoding performance for the ipsilateral cortical areas located ˜1.5-3 mm from the array. Interestingly, for the contralateral cortical areas, the decoding is still possible even though their activities are unlikely to be directly detected by the ECoG electrodes. In some embodiments, the successful decoding of contralateral cortical areas is mainly due to the fact that the activity of same functional cortical areas in both hemispheres are often correlated (FIGS. 8A-8B).

In some embodiments, the decoding results for the activity of individual cortical regions show that even with single ECoG channel, the decoding is possible (mean correlation performance between 0.35-0.65 for different cortical regions). By including more ECoG channels, an increase in decoding performance can be observed, but the performance starts to saturate after the inclusion of ˜10 ECoG channels (mean correlation performance between 0.6-0.75 for different cortical regions). In some embodiments, this is mainly because of the fact that the ECoG signals in adjacent channels are correlated due to the volume conduction in the brain tissue. It has been shown that the correlation between ECoG signals from adjacent channels at different frequency bands decreases as the distance increases. Even though the cross-channel correlation at high frequency bands can be lower than that at low frequency bands, it does not go below chance level even with a distance of ˜1.5 mm. However, the results empirically confirm that even though the ECoG signals from adjacent channels are highly correlated, they still differentially encode information about the cortical activities to some extent so that sequentially including more ECoG channels tends to increase the decoding performance. However, beyond a certain threshold adding more ECoG channels does not further increase the decoding performance.

For the decoding of cortex-wide brain activity, instead of attempting to directly reconstruct the activity of individual pixels, the disclosed technology can be implemented in some embodiments to perform PCA followed by spatial ICA on the cortical activity and later to decode IC scores to recover the cortex-wide activity at pixel level. The adoption of this approach is based on both scientific and computational considerations. First, the PCA effectively reduced the spatial dimensions, while preserving a large proportion of variance in cortical activity. Since the activity of each single pixel is noisy and unreliable, performing PCA reduced the noise, leading to a more reliable estimate of the true activity. Second, choosing the IC scores as network outputs greatly reduced the parameters in the output layer of the neural network model, prevented overfitting, and speeded up model training. Finally, the spontaneous cortex-wide brain activity is decomposed into a set of local and spatially organized cortical activation patterns, which is a biologically meaningful decomposition that matches the brain dynamics. This decomposition provides a good demixing of cortex-wide brain activity and enables a tractable mapping from ECoG responses, which can be learned by the decoding network model. Taken together, these results reveal that the activation of different cortical functional modules are associated with distinct multi-channel ECoG responses. By exploiting the mapping between the two modalities, a virtual imaging of cortex-wide brain activity is possible from locally recorded ECoG signals.

Fabrication of Graphene Array

Electrode arrays are fabricated on 4″ Silicon wafers spin coated with 20 μm-thick PDMS. 50 μm-thick PET (Mylar 48-02F-OC) is placed on the adhesive PDMS layer and used as the array substrate. 10 nm of chromium and 100 nm of gold are deposited onto the PET using a Denton 18 Sputtering System. The metal wires are patterned using photolithography and wet etching methods. Single-layer graphene is placed on the array area using a previously developed transfer process. The wafer is then soft baked for 5 minutes at 125° C. to better adhere graphene to the substrate. PMMA is removed via a 20-minute acetone bath at room temperature then rinsed with isopropyl alcohol and DI water for ten 1 min cycles. The graphene channels are patterned using AZ1512/PMGI bilayer photolithography then oxygen plasma etched (Plasma Etch PE100). A four-step cleaning method is performed on the array consisting of an AZ NMP soak, remover PG soak, acetone soak, and 10-cycle isopropyl alcohol/DI water rinse. 8 μm-thick SU-8 2005 is spun onto the wafer as an encapsulation layer and openings are created at the active electrical regions using photolithography. The array is then given a final 10-cycle isopropyl alcohol/DI water rinse to clean SU-8 residue and baked for twenty minutes at temperature progressing from 125° C. to 13 5° C.

Mice are group-housed in disposable plastic cages with standard bedding in a room with a reversed light cycle (12 h-12 h). Experiments are performed during the dark period. Both male and female healthy adult mice are used. Mice had no prior history of experimental procedures that may affect the results.

Surgery and Multimodal Experiments

Adult mice (6 weeks or older) are anesthetized with 1-2% isoflurane and injected with baytril (10 mg/kg) and buprenorphine (0.1 mg/kg) subcutaneously. A circular piece of scalp is removed to expose the skull. After cleaning the underlying bone using a surgical blade, a custom-built head-bar is implanted onto the exposed skull over the cerebellum (˜1 mm posterior to lambda) with cyanoacrylate glue and cemented with dental acrylic (Lang Dental). Two stainless-steel wires (791900, A-M Systems) are implanted into the cerebellum as ground/reference. A craniotomy (˜7 mm×8 mm) is made to remove most of the dorsal skull and the graphene array is placed on the surface of one hemisphere, covering somatosensory cortex (S1) and posterior parietal cortex (PPC). The exposed cortex and the array are covered with a custom-made curved glass window, which is further secured with an adhesive (e.g., Vetbond (3M)), cyanoacrylate glue and dental acrylic. Animals are fully awake before recordings.

The wide-field calcium imaging is performed using a commercial fluorescence microscope (Axio Zoom.V16, Zeiss, objective lens (1×, 0.25 NA)) and a CMOS camera (ORCA-Flash4.0 V2, Hamamatsu) through the intact skull as previously described. Images are acquired using HCImage Live (Hamamatsu) at 29.98 Hz, 512×512 pixels (field of view: 11 mm×11 mm, binning: 4, 16 bit).

The microelectrode array is connected to a custom-made connector board through a ZIF connector. The ECoG data is sampled with Intan RHD2132 amplifier and recorded using Intan RHD2000 system. The sampling frequency is 10 kHz. Three mice are recorded, each having 2-3 recording sessions. The length for each recording session is 1 hour.

ΔF/F Processing

To obtain the ΔF/F time series from the wide-field calcium imaging data, the 512×512 pixel images are first down-sampled to smaller images of 128×128 pixels. For each pixel, a dynamic fluorescence (F) baseline for a given time point can be defined as the 10th percentile value over 180 s around it. For the beginning and ending of each imaging block, the following and preceding 90-s window is used to determine the baseline, respectively. An 8th order 6 Hz Butterworth low-pass filter is applied to the ΔF/F activity of each pixel to remove the high frequency noise. The activity of each cortical region is obtained by averaging over the ΔF/F signals from all the pixels within the same cortical regions defined by the Allen Brain Atlas (FIG. 1C).

ECoG Processing

The raw ECoG signals are first passed through notch filters to eliminate the 60 Hz powerline contaminations and their higher harmonics at 120 Hz and 180 Hz. The signals are further filtered with multiple 6th order Butterworth band-pass filters designed for different frequency bands (δ: 1-4 Hz, θ: 4-7 Hz, α: 8-15 Hz, β: 15-30 Hz, γ: 31-59 Hz, H-γ: 61-200 Hz). The resulting signals are squared and smoothed by a Gaussian function with 100 ms time window to obtain an estimate of the instantaneous power. To prepare the input data for the decoding neural network, the ECoG power traces at different frequency bands are down-sampled to 29.98 Hz by interpolation to match the sampling rate of calcium imaging data. To suppress the potential artifacts in the ECoG signal, at each frequency band the power traces can be clipped with a threshold of 95 percentile.

Neural Network Models

The neural network model consists of a sequential stacking of a linear hidden layer, one bidirectional LSTM layer and a linear readout layer. The first linear layer is followed by batch normalization, ReLU activation, and dropout with a probability of 0.3. The LSTM layer is followed by batch normalization. The multichannel ECoG power at different frequency bands are used as inputs to the network. To decode the neural activity at each time step t, the ECoG power segments between [t−1.5 s, t+1.5 s] is used (90 time steps in total). The first linear layer had 16 neurons and the bidirectional LSTM had 8 hidden neurons. The same neural network model is used for the two decoding tasks except that the number of neurons in the final output layer differs based on the targeting output. To decode the ΔF/F activity of 12 cortical regions simultaneously, the output neuron number is set to 12. To decode the cortex-wide brain activity, the output neuron number is set to 10 to generate the scores for the 10 ICs.

In some embodiments, the neural network model is implemented in Pytorch. The model parameters are trained through Adam optimizer with learning rate=1e−4, beta1=0.9, beta2=0.999, epsilon=1e−8. The batch size is 128 and the training usually converged within ˜30 epochs. For both the tasks, the mean squared error is chosen as the loss function. The disclosed technology can be implemented in some embodiments to perform 10-fold cross-validation where each 1 h recording session is chunked into ten segments, each lasting for 6 min. The neural network model is trained on 9/10 of the data segments and tested on a different held-out segment that is unseen during the training. To evaluate the model performance, correlation between the decoded and ground truth data for each held-out set is averaged. For each 1 h recording session, the network is trained and tested separately. Then, for each mouse, the correlation is further averaged across the recording sessions to give the performance for that mouse (FIGS. 3B3D, FIGS. 4E and 4F).

Statistical Tests

All statistical analyses are performed in MATLAB. Statistical tests are two-tailed and significance is defined by alpha pre-set to 0.05. All the statistical tests are described in the figure legends. Multiple comparisons are corrected for by Benjamini-Hochber corrections.

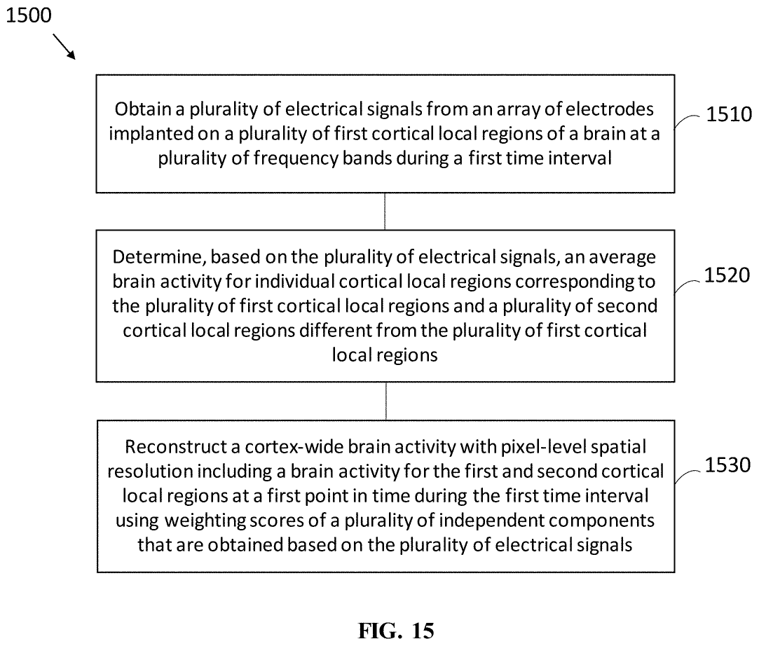

FIG. 15 shows an example method for reconstructing a cortex-wide brain activity based on some embodiments of the disclosed technology.

In some implementations, a method 1500 includes, at 1510, obtaining a plurality of electrical signals from an array of electrodes implanted on a plurality of first cortical local regions of a brain at a plurality of frequency bands during a first time interval, at 1520, determining, based on the plurality of electrical signals, an average brain activity for individual cortical local regions corresponding to the plurality of first cortical local regions and a plurality of second cortical local regions different from the plurality of first cortical local regions, and at 1530, reconstructing a cortex-wide brain activity with pixel-level spatial resolution including a brain activity for the first and second cortical local regions at a first point in time during the first time interval using weighting scores of a plurality of independent components that are obtained based on the plurality of electrical signals.

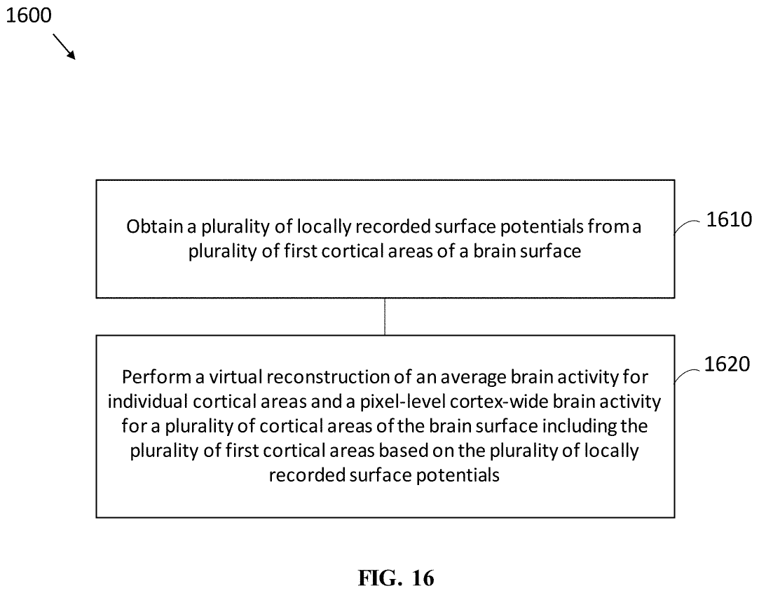

FIG. 16 shows an example of a virtual reconstruction method of a cortex-wide brain activity based on some embodiments of the disclosed technology.

In some implementations, a method 1600 includes, at 1610, obtaining a plurality of locally recorded surface potentials from a plurality of first cortical areas of a brain surface, and at 1620, performing a virtual reconstruction of an average brain activity for individual cortical areas and a pixel-level cortex-wide brain activity for a plurality of cortical areas of the brain surface including the plurality of first cortical areas based on the plurality of locally recorded surface potentials.

Therefore, various implementations of features of the disclosed technology can be made based on the above disclosure, including the examples listed below.

Example 1. A method comprising: obtaining a plurality of electrical signals from an array of electrodes implanted on a plurality of first cortical local regions of a brain at a plurality of frequency bands during a first time interval; determining, based on the plurality of electrical signals, an average brain activity for individual cortical local regions corresponding to the plurality of first cortical local regions and a plurality of second cortical local regions different from the plurality of first cortical local regions; and reconstructing a cortex-wide brain activity with pixel-level spatial resolution including a brain activity for the first and second cortical local regions at a first point in time during the first time interval using weighting scores of a plurality of independent components that are obtained based on the plurality of electrical signals.

Example 2. The method of example 1, further comprising generating a virtual image of the cortex-wide brain activity using the reconstructed cortex-wide brain activity.

Example 3. The method of example 1, wherein the average brain activity for individual cortical local regions is determined by averaging changes in fluorescence intensity from a plurality of image pixels within each cortical local region.

Example 4. The method of example 1, wherein the plurality of electrical signals includes electrocorticography (ECoG) signals.

Example 5. The method of example 4, wherein determining the average activity for individual cortical local regions is based on an ECoG power of a plurality of frequency bands from a plurality of electrical signal channels.

Example 6. The method of example 1, wherein the weighting scores of a plurality of independent components are determined using a spatial independent component analysis.

Example 7. The method of example 1, wherein the first time interval starts earlier than the first point in time and ends later than the first point in time.

Example 8. The method of example 1, wherein the plurality of first cortical local regions includes at least one of: secondary motor cortex; primary motor cortex; primary somatosensory cortex; posterior parietal cortex; retrosplenial cortex; or visual cortex.

Example 9. The method of example 1, wherein the electrodes include transparent graphene microelectrodes.

Example 10. The method of example 9, wherein obtaining the plurality of electrical signals includes performing a wide-field calcium imaging.

Example 11. The method of example 1, wherein reconstructing the cortex-wide brain activity includes using a neural network algorithm that includes a sequential stacking of a linear hidden layer, a bidirectional long short-term memory (Bi-LSTM) layer, and a linear readout layer.

Example 12. The method of example 11, wherein the electrodes include transparent graphene microelectrodes, wherein the graphene microelectrodes are used to collect training data for the neural network algorithm.

Example 13. A method comprising: obtaining a plurality of locally recorded surface potentials from a plurality of first cortical areas of a brain surface; and performing a virtual reconstruction of an average brain activity for individual cortical areas and a pixel-level cortex-wide brain activity for a plurality of cortical areas of the brain surface including the plurality of first cortical areas based on the plurality of locally recorded surface potentials.

Example 14. The method of example 13, wherein the plurality of locally recorded surface potentials includes electrocorticography (ECoG) signals.

Example 15. The method of example 13, wherein obtaining the plurality of locally recorded surface potentials includes obtaining a plurality of locally recorded surface potentials from an array of electrodes implanted on the plurality of cortical areas of the brain surface.

Example 16. The method of example 15, wherein the electrodes include transparent graphene microelectrodes.

Example 17. The method of example 13, wherein performing the virtual reconstruction includes virtual imaging of an averaged spontaneous activity from the plurality of first cortical areas of the brain surface.

Example 18. The method of example 13, wherein performing the virtual reconstruction includes using a neural network algorithm that includes a sequential stacking of a linear hidden layer, a bidirectional long short-term memory (Bi-LSTM) layer, and a linear readout layer.

Example 19. The method of example 13, wherein obtaining the plurality of locally recorded surface potentials includes locally recording surface potentials from an array of electrodes implanted on the plurality of cortical areas of the brain surface at a plurality of time frames.

Example 20. The method of example 13, wherein the plurality of first cortical areas includes at least one of: secondary motor cortex; primary motor cortex; primary somatosensory cortex; posterior parietal cortex; retrosplenial cortex; or visual cortex.

Example 21. A device comprising: an array of electrodes configured to be implanted on a plurality of first cortical local regions of a brain; a memory to store instructions for performing a virtual reconstruction of an activity of the brain; and a processor in communication with the memory, wherein the instructions upon execution by the process cause the processor to: obtain a plurality of electrical signals from the array of electrodes implanted on a plurality of first cortical local regions of the brain at a plurality of frequency bands during a first time interval; determine, based on the plurality of electrical signals, an average brain activity for individual cortical local regions corresponding to the plurality of first cortical local regions and a plurality of second cortical local regions different from the plurality of first cortical local regions; and reconstruct a cortex-wide brain activity with pixel-level spatial resolution including a brain activity for the first and second cortical local regions at a first point in time during the first time interval using weighting scores of a plurality of independent components that are obtained based on the plurality of electrical signals.

Example 22. The device of example 21, further comprising an imaging device configured to generate a virtual image of the cortex-wide brain activity using the reconstructed cortex-wide brain activity.

Example 23. The device of example 21, wherein the average brain activity for individual cortical local regions is determined by averaging changes in fluorescence intensity from a plurality of image pixels within each cortical local region.

Example 24. The device of example 21, wherein the plurality of electrical signals includes electrocorticography (ECoG) signals.

Example 25. The device of example 21, wherein the first time interval starts earlier than the first point in time and ends later than the first point in time.

Example 26. The device of example 21, wherein the plurality of first cortical local regions includes at least one of: secondary motor cortex; primary motor cortex; primary somatosensory cortex; posterior parietal cortex; retrosplenial cortex; or visual cortex.

Example 27. The device of example 21, wherein the electrodes include transparent graphene microelectrodes.

Example 28. The device of example 21, wherein reconstructing the cortex-wide brain activity includes using a neural network algorithm that includes a sequential stacking of a linear hidden layer, a bidirectional long short-term memory (Bi-LSTM) layer, and a linear readout layer.

Implementations of the subject matter and the functional operations described in this patent document can be implemented in various systems, digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible and non-transitory computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them. The term “data processing unit” or “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of nonvolatile memory, media and memory devices, including by way of example semiconductor memory devices, e.g., EPROM, EEPROM, and flash memory devices. The processor and the memory can be supplemented by, or incorporated in, special purpose logic circuitry.

It is intended that the specification, together with the drawings, be considered exemplary only, where exemplary means an example. As used herein, the singular forms“a”, “an” and “the” are intended to include the plural forms as well, unless the context clearly indicates otherwise. Additionally, the use of “or” is intended to include “and/or”, unless the context clearly indicates otherwise.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a sub combination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

What is claimed is:

1. A method comprising:
obtaining a plurality of electrical signals from an array of electrodes implanted on a plurality of first cortical local regions of a brain at a plurality of frequency bands during a first time interval;
determining, based on the plurality of electrical signals, an average brain activity for individual cortical local regions corresponding to the plurality of first cortical local regions and a plurality of second cortical local regions different from the plurality of first cortical local regions; and
reconstructing a cortex-wide brain activity with pixel-level spatial resolution including a brain activity for the first and second cortical local regions at a first point in time during the first time interval using weighting scores of a plurality of independent components that are obtained based on the plurality of electrical signals.

2. The method of claim 1, further comprising generating a virtual image of the cortex-wide brain activity using the reconstructed cortex-wide brain activity.

3. The method of claim 1, wherein the average brain activity for individual cortical local regions is determined by averaging changes in fluorescence intensity from a plurality of image pixels within each cortical local region.

4. The method of claim 1, wherein the plurality of electrical signals includes electrocorticography (ECoG) signals.

5. The method of claim 4, wherein determining the average activity for individual cortical local regions is based on an ECoG power of a plurality of frequency bands from a plurality of electrical signal channels.

6. The method of claim 1, wherein the weighting scores of a plurality of independent components are determined using a spatial independent component analysis.

7. The method of claim 1, wherein the first time interval starts earlier than the first point in time and ends later than the first point in time.

8. The method of claim 1, wherein the plurality of first cortical local regions includes at least one of: secondary motor cortex; primary motor cortex; primary somatosensory cortex; posterior parietal cortex; retrosplenial cortex; or visual cortex.

9. The method of claim 1, wherein the electrodes include transparent graphene microelectrodes.

10. The method of claim 9, wherein obtaining the plurality of electrical signals includes performing a wide-field calcium imaging.

11. The method of claim 1, wherein reconstructing the cortex-wide brain activity includes using a neural network algorithm that includes a sequential stacking of a linear hidden layer, a bidirectional long short-term memory (Bi-LSTM) layer, and a linear readout layer.

12. The method of claim 11, wherein the electrodes include transparent graphene microelectrodes, wherein the graphene microelectrodes are used to collect training data for the neural network algorithm.

13. A method comprising:
obtaining a plurality of locally recorded surface potentials from a plurality of first cortical areas of a brain surface; and
performing a virtual reconstruction of an average brain activity for individual cortical areas and a pixel-level cortex-wide brain activity for a plurality of cortical areas of the brain surface including the plurality of first cortical areas based on the plurality of locally recorded surface potentials.

14. The method of claim 13, wherein the plurality of locally recorded surface potentials includes electrocorticography (ECoG) signals.

15. The method of claim 13, wherein obtaining the plurality of locally recorded surface potentials includes obtaining a plurality of locally recorded surface potentials from an array of electrodes implanted on the plurality of cortical areas of the brain surface.

16. The method of claim 15, wherein the electrodes include transparent graphene microelectrodes.

17. The method of claim 13, wherein performing the virtual reconstruction includes virtual imaging of an averaged spontaneous activity from the plurality of first cortical areas of the brain surface.

18. The method of claim 13, wherein performing the virtual reconstruction includes using a neural network algorithm that includes a sequential stacking of a linear hidden layer, a bidirectional long short-term memory (Bi-LSTM) layer, and a linear readout layer.

19. The method of claim 13, wherein obtaining the plurality of locally recorded surface potentials includes locally recording surface potentials from an array of electrodes implanted on the plurality of cortical areas of the brain surface at a plurality of time frames.

20. The method of claim 13, wherein the plurality of first cortical areas includes at least one of: secondary motor cortex; primary motor cortex; primary somatosensory cortex; posterior parietal cortex; retrosplenial cortex; or visual cortex.

21. A device comprising:
an array of electrodes configured to be implanted on a plurality of first cortical local regions of a brain;
a memory to store instructions for performing a virtual reconstruction of an activity of the brain; and
a processor in communication with the memory, wherein the instructions upon execution by the processor cause the processor to:
obtain a plurality of electrical signals from the array of electrodes implanted on a plurality of first cortical local regions of the brain at a plurality of frequency bands during a first time interval;
determine, based on the plurality of electrical signals, an average brain activity for individual cortical local regions corresponding to the plurality of first cortical local regions and a plurality of second cortical local regions different from the plurality of first cortical local regions; and
reconstruct a cortex-wide brain activity with pixel-level spatial resolution including a brain activity for the first and second cortical local regions at a first point in time during the first time interval using weighting scores of a plurality of independent components that are obtained based on the plurality of electrical signals.

22. The device of claim 21, further comprising an imaging device configured to generate a virtual image of the cortex-wide brain activity using the reconstructed cortex-wide brain activity.

23. The device of claim 21, wherein the average brain activity for individual cortical local regions is determined by averaging changes in fluorescence intensity from a plurality of image pixels within each cortical local region.

24. The device of claim 21, wherein the plurality of electrical signals includes electrocorticography (ECoG) signals.

25. The device of claim 21, wherein the first time interval starts earlier than the first point in time and ends later than the first point in time.

26. The device of claim 21, wherein the plurality of first cortical local regions includes at least one of: secondary motor cortex; primary motor cortex; primary somatosensory cortex; posterior parietal cortex; retrosplenial cortex; or visual cortex.

27. The device of claim 21, wherein the electrodes include transparent graphene microelectrodes.

28. The device of claim 21, wherein reconstructing the cortex-wide brain activity includes using a neural network algorithm that includes a sequential stacking of a linear hidden layer, a bidirectional long short-term memory (Bi-LSTM) layer, and a linear readout layer.
